(19) United States
(12) Patent Application Publication
(10) Pub. No.: US 2003/0095982 A1
(43) Pub. Date: May 22, 2003

(54) Fraction of Cordyceps sinensis and method of isolation thereof

(76) Inventor: Ching-Yuang Lin, Taipei (TW)

Correspondence Address: BACON & THOMAS, PLLC, 625 SLATERS LANE, FOURTH FLOOR, ALEXANDRIA, VA, 22314

(21) Appl. No.: 10/216,847

(22) Filed: Aug. 13, 2002

Related U.S. Application Data
(63) Continuation of application No. 09/820,656, filed on Mar. 30, 2001, now abandoned.

(51) Int. Cl. A61K 35/84

(52) U.S. Cl. 424/195.15; 424/93.5

(57) ABSTRACT

A method for identifying and isolating the active fractions in fungus Cordyceps sinensis. It covers a findings of the structure of an active compound that is present in the active fractions, an isolation method that can be used to extract the active fractions and a specific active compound, and the use of F8, one specific active compound, that is present in the active fractions thus isolated, to improve the clinical symptoms of bronchial hyperresponsiveness and pulmonary injury in OA induced BNR model with enhancing Th1 cytokines suppressing Th2 and iNOS cytokines mRNA expression. This work has important pharmacological implications for the prevention and treatment of bronchial asthma in humans.

### BACKGROUND OF THE INVENTION

1. Field of the Invention

This invention relates to a method of isolating the fractions of Cordyceps sinensis and extracting the constituent F8 therein, which can suppress platelets activating factor (PAF) inducing rabbit platelete aggregation in vitro, improving pulmonary function of animals with ovalbumin (OA) induced bronchial hyperresponsiveness in vivo, enhancing Th1 cytokines that inhibit Th2 cytokines and induce nitric oxide synthase (iNOS) genes expression in vivo in brown Norway rats (BNR) with OA induced bronchial hyperresponsiveness, alleviating the bronchial hyperresponsiveness of bronchial asthma and histopathologically preventing chronic inflammatory injury.

2. Description of the Prior Art

Recent studies have demonstrated the multiple pharmacological actions of Cordyceps sinensis (Claricipitaceae), which is a fungus that develops stroma and is found on the larvae of the lipidoptera caterpillar. The pharmacological actions include:

1. The Respiratory System:

The extract of Cordyceps sinensis can obviously dilate bronchial smooth muscle of guinea pigs in vitro to relief asthma attack. It can also enhance the ability of mice to tolerate hypoxic insult in normal pressure, to relax tracheal wall directly, to prevent pulmonary emphysema induced by inhalation of aerosolized CsCl, to protect tracheal epithelium and enhance anti-injury ability of the respiratory tract.

2. Liver Function:

The extract of Cordyceps sinensis can enhance phagocytotic ability of macrophage, blood flow volume of liver and elevate the activity of collagenase in liver tissue. In the treatment of liver cirrhosis due to hepatitis, it has the effect of anti-hepatic fibrosis.

3. The Immune System:

The extract of Cordyceps sinensis can enhance the activity of nature killer cells in both normal and leukemic patients, enhance expression of lymphocyte surface antigen CD 16, and elevate the rate of binding with K562 cells. This can enhance cellular immunity, elevate the ratio of Th/Ts, and decrease the immunosuppressive effect due to steroid and cyclophosphamide prescriptions.

4. The Cardiovascular System:

The extract of Cordyceps sinensis can slow heart rates in anesthetized rats and guinea pigs. It also reduces resistance and pressure in arteries, brain and peripheral vascular system. In addition, Cordyceps sinensis has the effects of anti-hypoxia, inhibiting monoamine oxidase (MAO) effect, relaxing vascular smooth muscles, and dilating vessel activity.

However, the above pharmacological functions were never studied with purified compounds from Cordyceps sinensis. None of the researches conducted on alleviating the pulmonary function, histological symptoms and immunological derivation were based on any animal model of bronchial asthma.

Clinically, bronchial asthma presented with paroxysmal expiratory wheezing is a chronic obstructive lower respiratory tract disease. The prevalence in our country is about 10%. Complications and increasing mortality and morbidity even to death are noted when treatments are inadequately prescribed. The tendency of gradually increasing mortality on asthma in noted all over the world. Except for bronchodilators and adrenal corticosteroids which can temporally improve bronchial constriction, there is no evidence of any agent which can improve broncheal and pulmonary chronic inflammation and pulmonary function.

Bronchial asthma is a multifactorial disease. Patients have atopic allergies when they are sensitized by inhalating allergens and B cells are activated that produce specific IgE antibodies. There are many mast cells in the epithelium and submucosal layer of the respiratory tract. IgE receptors (Fcε R1) with high affinity are noted in the surface of mast cells. When they react with specific IgE, sensitized status is established. If allergens invade once more and cross-like with two IgE antibodies binding with IgE receptors in mast cells surface, the signals will transduct into mast cells. Cell membrane will produce and release platelet activating factor (PAF), leukotrienes etc. On the other hand, intracytoplasmic granules including histamine, proteinase and chemotaxtic factors with be released. So in addition to immediate bronchial constriction, many inflammatory cells are attracted. The former can be antagonized by a bronchodilator. However, the latter will result in infiltration of many monocytes, eosinophils and basophils and releasing of many cytokines, growth factors and proteinase etc. It makes smooth muscle constrict, vessel permeability increase, capillary plasma exudate, respiratory secretion produce, epithelium and basement membrane cells slough, bronchial tract suffer permanently injury and pulmonary function gradually decrease. Therefore inflammatory response is the major mechanism of complications and death in asthma. Among the inflammatory responses, PAF has the strongest and longest effect on eosinophil and neutrophil infiltration. Its effect can prolong 4 weeks with self-amplification effect. This means that the eosinophils and neutrophils attracted by PAF can release PAF again and attract more eosinophil and neutrophil infiltration, further injuring bronchus and lung.

### SUMMARY OF THE INVENTION

The main object of the invention is to find certain fractions and compounds produced by Cordyceps sinensis that can be used to inhibit PAF induced rabbit platelets aggregation in vitro, improve the pulmonary function and histological changes in BNR asthma animal model, and enhance Th1 cells cytokines that inhibit Th2 cytokines iNOS gene expression in vivo.

A further object of the invention is to provide a method for obtaining certain fractions and isolating the active compounds.

To reach the above-mentioned objectives, a method for inhibiting PAF induced rabbit platelete aggregation in vitro is adopted for investigating the inhibition of PAF function and the BNR animal asthma model for the investigation of bronchial hyperresponsiveness, histopathological change, and Th1, Th2 cytokines and iNOS gene expression. Some methods are used, such as, inhibited PAF inducing rabbit platelet aggregation in vitro, acute toxicity test, and animal model of bronchial hyperresponsiveness, to find particular fractions and compounds that may be used in the treatment of the disease.

Other objects and the features of this invention can be understood by reading the following paragraphs of the detailed description and accompanying tables and figures.

### BRIEF DESCRIPTION OF THE DRAWINGS

FIG. 1 Flow chart for preparation of active fraction F-4 and isolation of active compound F8 from the fruiting bodies of the fungus Cordyceps sinensis.

FIG. 2 Chemical structure of compound F8. (24R)-ergosta-7,22-diene-3β, 5α, 6β,-triol

FIG. 3 Shows the proton nuclear magnetic resonance (¹H-NMR) spectrum of F8.

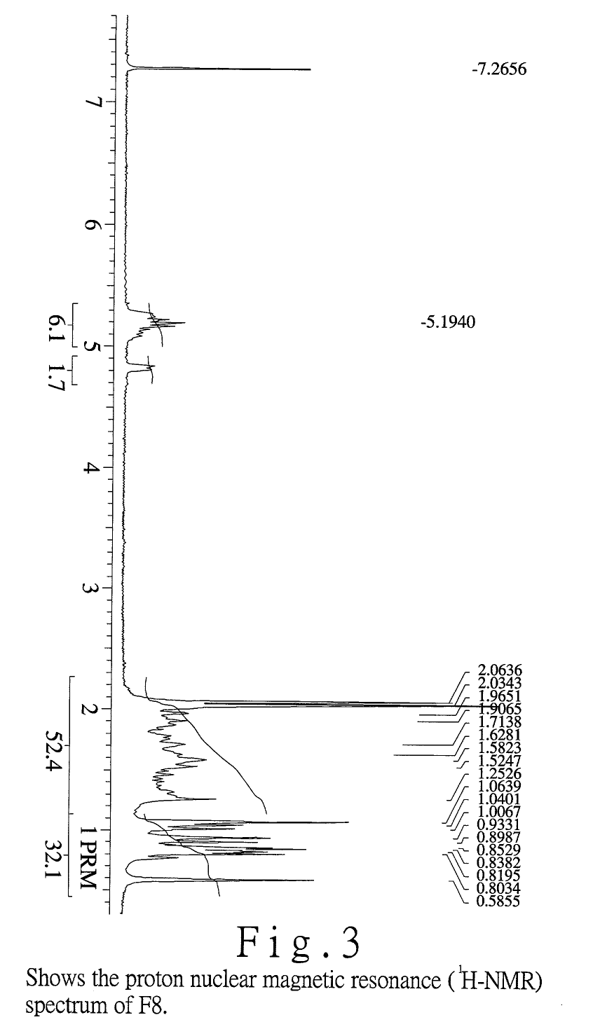

FIG. 4a Shows the methylation product of F8 of the proton nuclear magnetic resonance (¹H-NMR) spectrum.

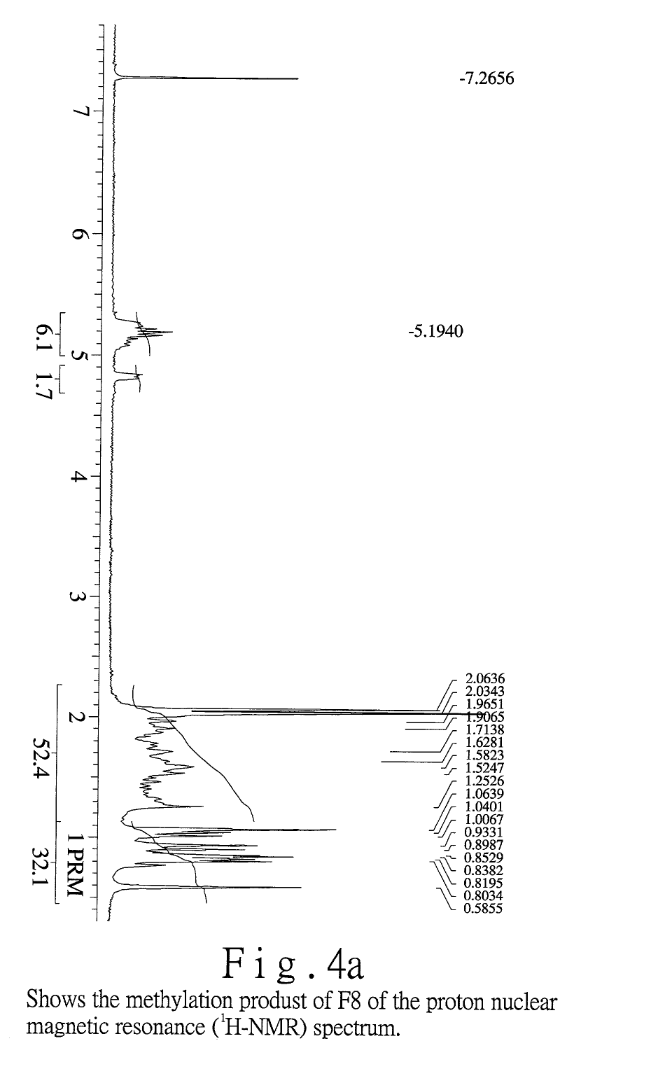

FIG. 4b Shows the proton nuclear magnetic resonance (¹H-NMR) spectrum range from δ 0.4 to 2.2 PPM of FIG. 4a.

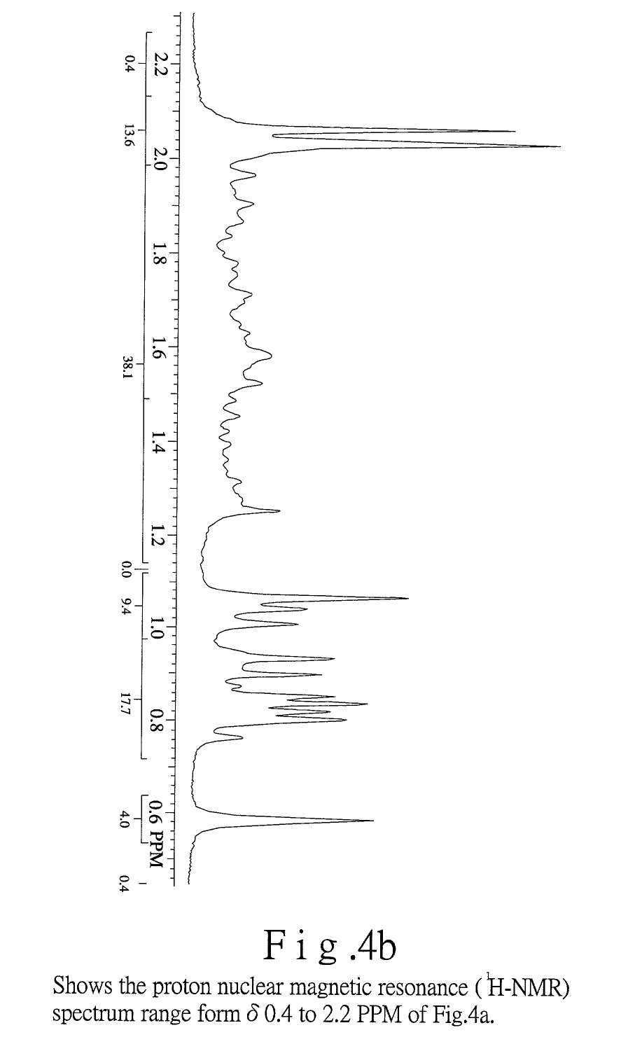

FIG. 5 Shows the ¹³carbon nuclear magnetic resonance (¹³C-NMR) spectrum of F8.

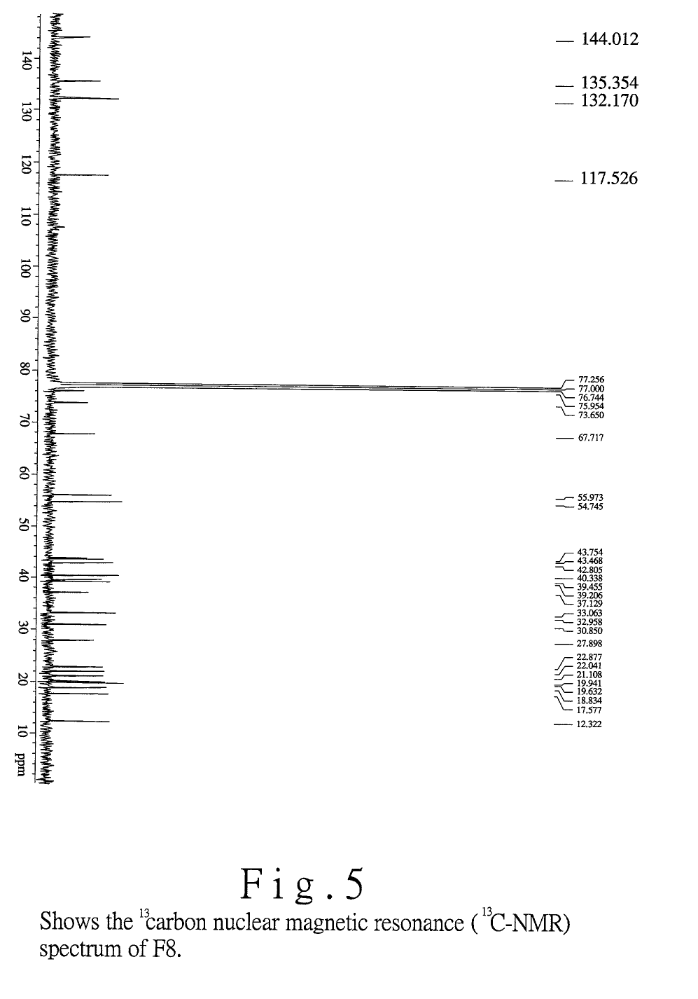

FIG. 6 Dose response curve of MFEF 50% TLC in BNR. The percentage of baseline MFEF 50% in group II was significantly lower then group I when Ach doses higher then 25 μg/kg were given (Group I: pretreated with F-4 before OA inhalation provocation; Group II: OA-treated; Group III: normal controls).

FIG. 7 Dose response curve of MFEF 25% TLC in BNR. The percentage of baseline MFEF 25% in group II was significantly lower then group I when Ach doses higher then 25 μg/kg were given (Group I: pretreated with F-4 before OA inhalation provocation; Group II: OA-treated; Group III: normal controls).

FIG. 8 Dose response curve of MFEF 50% TLC in guinea pigs.

The percentage of baseline MFEF 50% in group II was significantly lower then group I when Ach doses higher then 25 μg/kg were given (Group 1: pretreated with F-4 before OA inhalation provocation; Group II: OA-treated; Group III: normal controls).

FIG. 9 Dose response curve of MFEF 25% TLC in guinea pigs.

The percentage of baseline MFEF 25% in group II was significantly lower then group I when Ach doses higher then 25 μg/kg were given (Group I: pretreated with F-4 before OA inhalation provocation; Group II: OA-treated; Group III: normal controls).

FIG. 10 Dose response curve of MFEF 50% TLC in BALB/c mice.

The percentage of baseline MFEF 50% in group II was significantly lower then group I when Ach doses higher then 25 μg/kg were given (Group i: pretreated with F-4 before OA inhalation provocation; Group II: OA-treated; Group III: normal controls).

FIG. 11 Dose response curve of MFEF 25% TLC in BALB/c mice.

The percentage of baseline MFEF 50% in group II was significantly lower then group I when Ach doses higher then 25 μg/kg were given (Group I: pretreated with F-4 before OA inhalation provocation; Group II: OA-treated; Group III: normal controls).

FIG. 12 Dose response curve of MFEF 50% TLC in BNR. The percentage of baseline MFEF 50% in group II was significantly lower then group I when Ach doses higher then 25 μg/kg were given (Group I: F-4 is administered after OA inhalation provocation; Group II: OA-treated; Group III: normal controls).

FIG. 13 Dose response curve of MFEF 25% TLC in BNR. The percentage of baseline MFEF 25% in group II was significantly lower then group I when Ach doses higher then 25 μg/kg were given (Group I: F-4 is administered after OA inhalation provocation; Group II: OA-treated; Group III: normal controls).

FIG. 14 Dose response curve of MFEF 50% TLC in BNR. The percentage of baseline MFEF 50% in group II was significantly lower then group I when Ach doses higher then 25 μg/kg were given (Group I: pretreated with F8 before OA inhalation provocation; Group II: OA-treated; Group III: normal controls).

FIG. 15 Dose response curve of MFEF 25% TLC in BNR. The percentage of baseline MFEF 25% in group II was significantly lower then group I when Ach doses higher then 25 μg/kg were given (Group I: pretreated with F8 before OA inhalation provocation; Group II: OA-treated; Group III: normal controls).

FIG. 16 Dose response curve of MFEF 50% TLC in BALB/c mice. The percentage of baseline MFEF 50% in group II was significantly lower then group I when Ach doses higher then 25 μg/kg were given (Group I: pretreated with F8 before OA inhalation provocation; Group II: OA-treated; Group III: normal controls).

FIG. 17 Th2 gene expression in lung tissue of BNR. N, non-treatment; OA, ovalbumin inducing asthma; OA+F-4, prophylactic treatment of F-4 and ovalbumin inducing asthma. N=6.

FIG. 18 Th2 gene expression in lung tissue of BNR. N, non-treatment; OA, ovalbumin inducing asthma; OA+F-4, ovalbumin inducing asthma and therapeutic treatment of F-4. N=6.

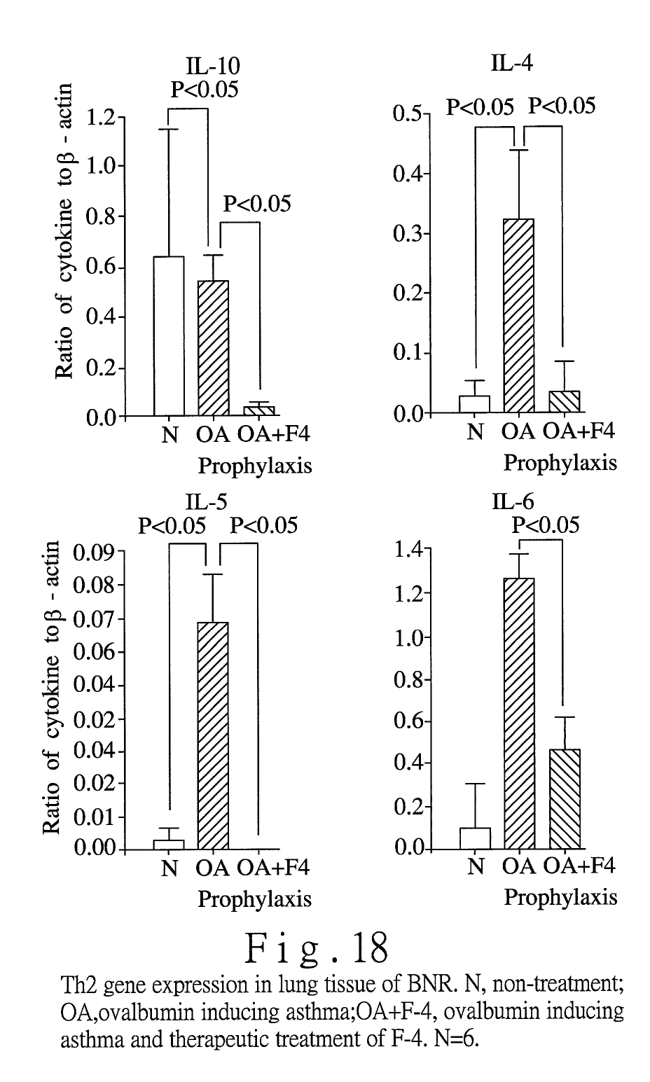

FIG. 19 Th2 gene expression in lung tissue of BNR. N, non-treatment; OA, ovalbumin inducing asthma; OA+F8, prophylactic treatment of F8 and ovalbumin inducing asthma. N=2.

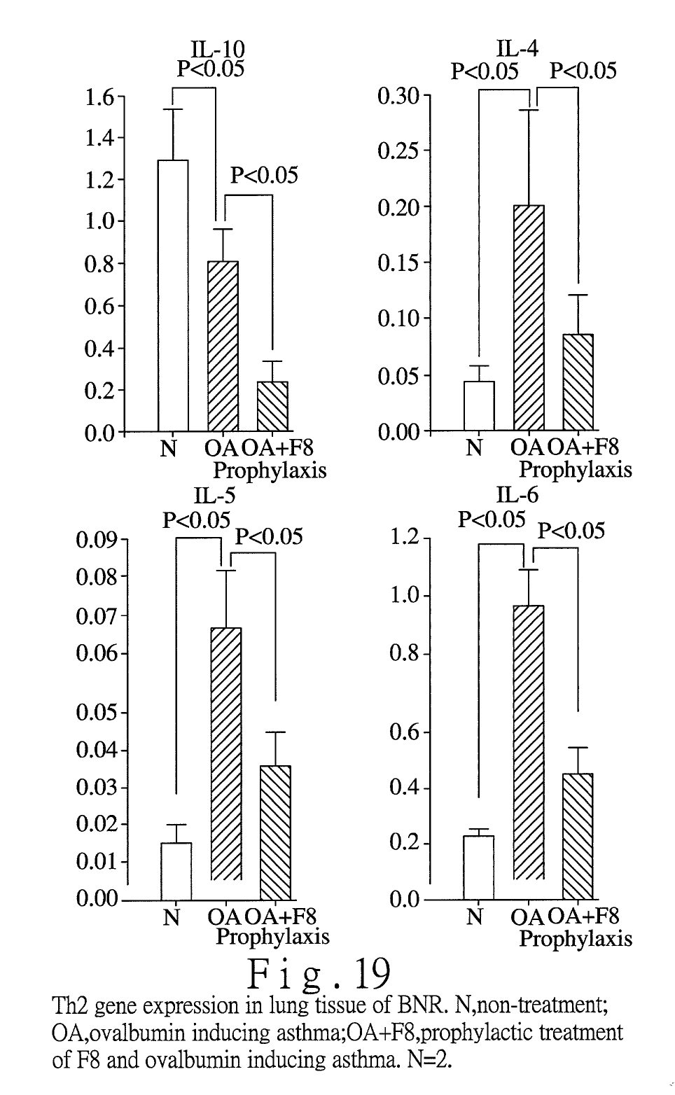

FIG. 20 Th2 gene expression in lung tissue of BNR. N, non-treatment; OA, ovalbumin inducing asthma; OA+F8, ovalbumin inducing asthma and therapeutic treatment of F8. n=2

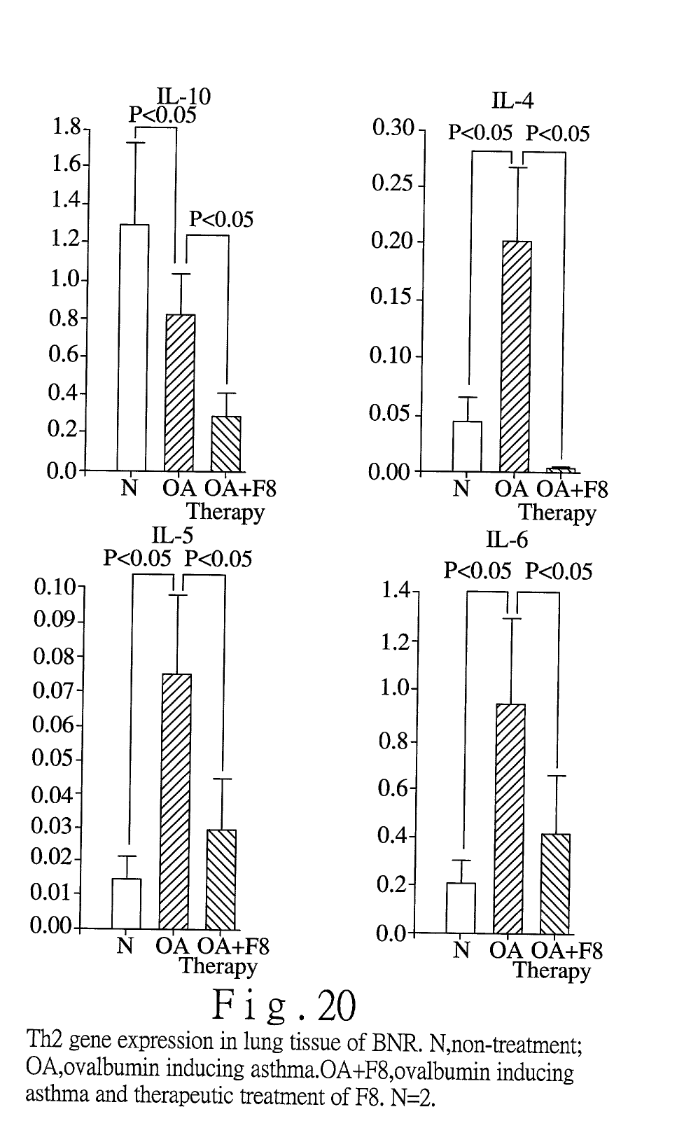

FIG. 21 IFN-γ gene expression in lung tissue of BNR. N, non-treatment; OA, ovalbumin inducing asthma; OA+F-4, prophylactic treatment of F-4 and ovalbumin inducing asthma. N=6.

FIG. 22 IFN-γ gene expression in lung tissue of BNR. N, non-treatment; OA, ovalbumin inducing asthma; OA+F-4, ovalbumin inducing asthma and therapeutic treatment of F-4. N=6.

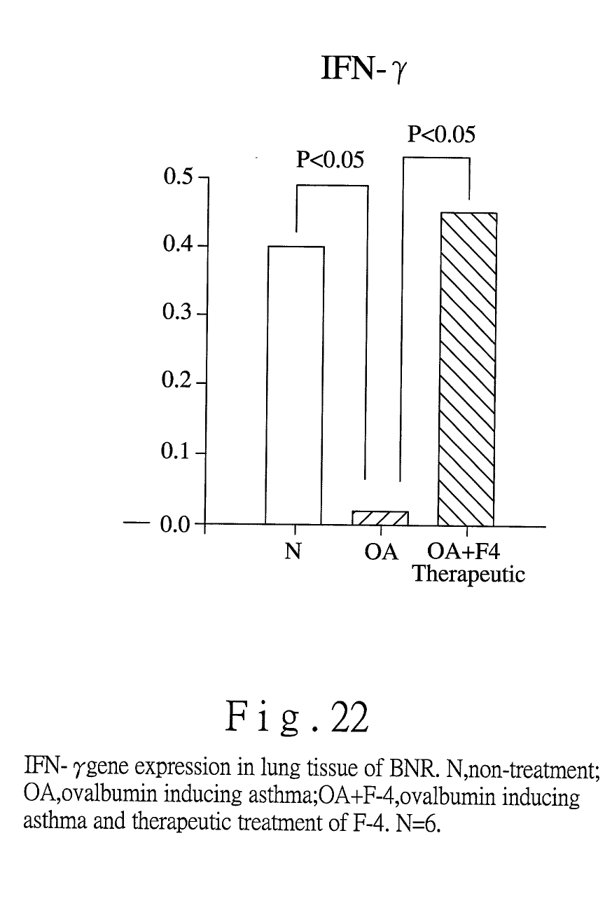

FIG. 23 IFN-γ gene expression in lung tissue of BNR. N, non-treatment; OA, ovalbumin inducing asthma; OA+F8, prophylactic treatment of F8 and ovalbumin inducing asthma. N=2.

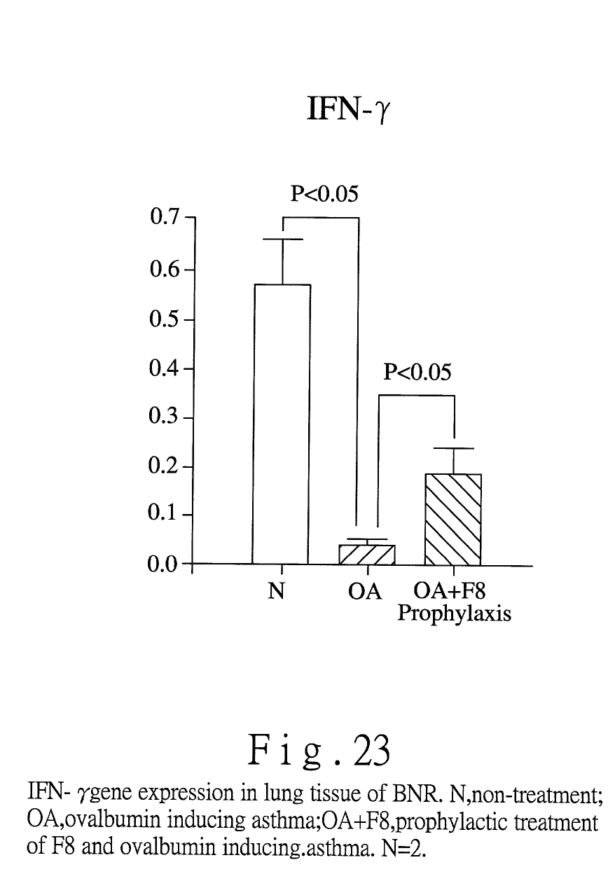

FIG. 24 IFN-γ gene expression in lung tissue of BNR. N, non-treatment; OA, ovalbumin inducing asthma; OA+F8, ovalbumin inducing asthma and therapeutic treatment of F8. n=2

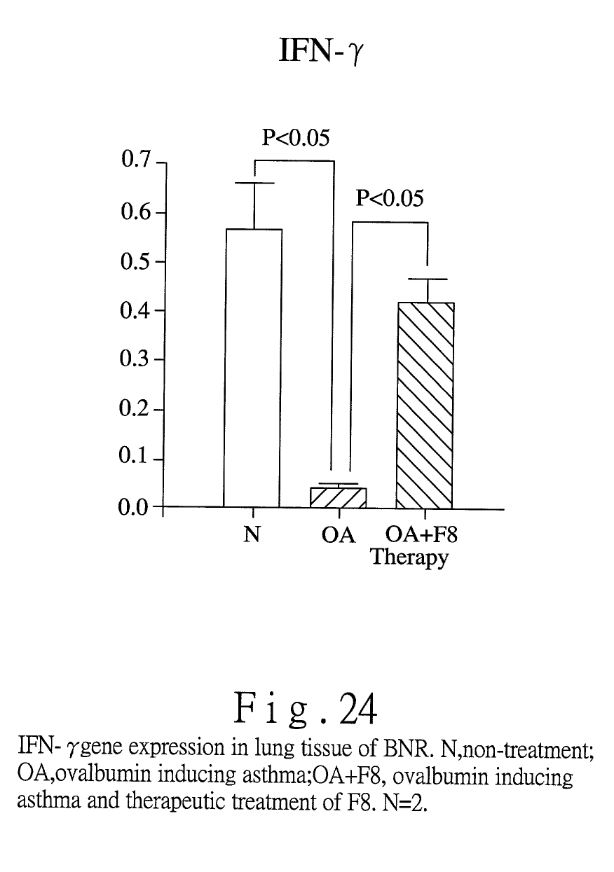

FIG. 25 iNOS gene expression in lung tissue of BNR. N, non-treatment; OA, ovalbumin inducing asthma; OA+F-4, prophylactic treatment of F-4 and ovalbumin inducing asthma. N=6.

FIG. 26 iNOS gene expression in lung tissue of BNR. N, non-treatment; OA, ovalbumin inducing asthma; OA+F-4, ovalbumin inducing asthma and therapeutic treatment of F-4. N=6.

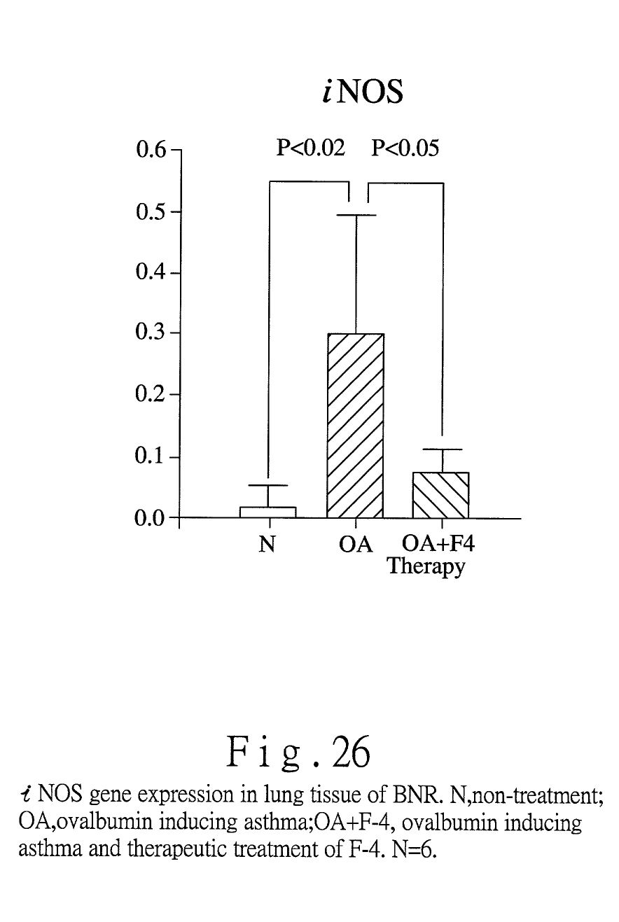

FIG. 27 iNOS gene expression in lung tissue of BNR. N, non-treatment; OA, ovalbumin inducing asthma; OA+F8, prophylactic treatment of F8 and ovalbumin inducing asthma. N=2.

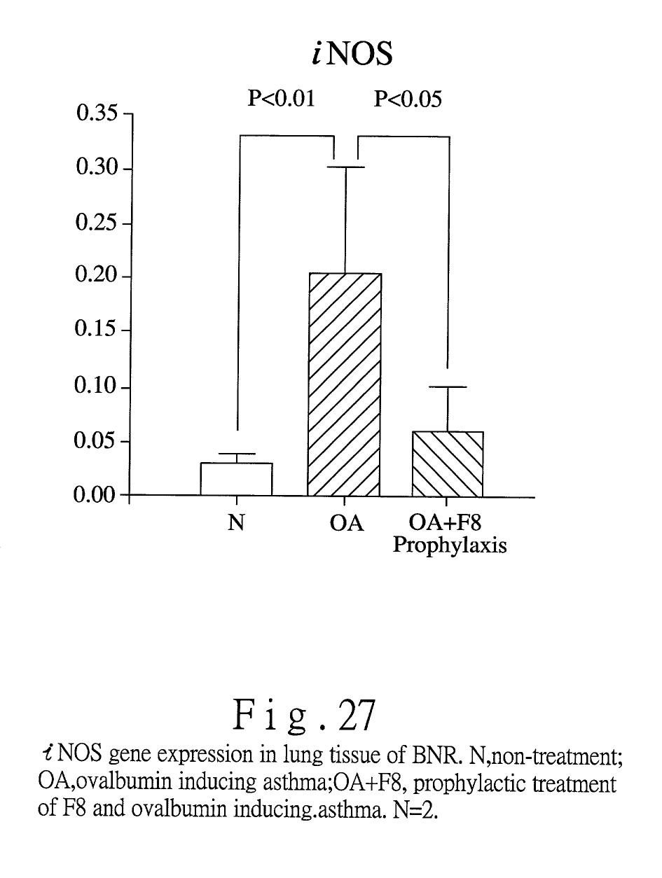

FIG. 28 iNOS gene expression in lung tissue of BNR. N, non-treatment; OA, ovalbumin inducing asthma; OA+F8, ovalbumin inducing asthma and therapeutic treatment of F8. n=2

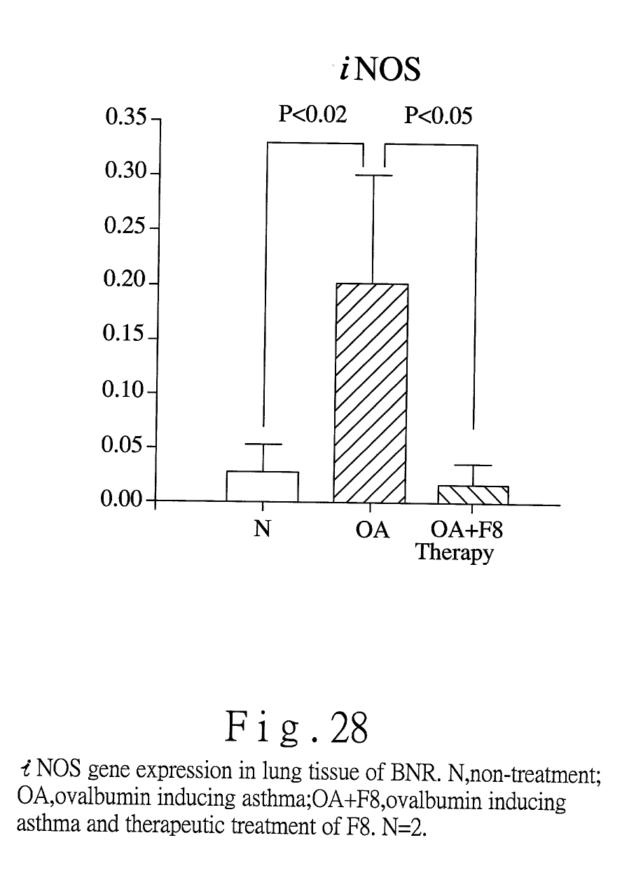

FIG. 29 EMSA analysis of nuclear NF-K B binding proteins in different lung tissue. The arrow indicate the position of specific protein-probe complex and the position of free probe. Nuclear proteins were extracted from lung tissue of BNR treated with none (lane 1 and 8), OA (lane 2 and 3), OA plus F-4 (prophylaxis)(lane 4, 6 and 7) and OA plus F8 (prophylaxis)(lane 5). 500 ng of cold NF-κ B probe was added for specific competition (lane 6). 1 μg each mNF-κB probe, pBR322 and λ/Hind III were added for nonspecific competition (lane 7).

FIG. 30 Super-shift assay analysis of nuclear NF-κ B binding proteins in different lung tissue. The arrows indicate the position of anti-p50 antibody-protein-DNA probe complex, the position of specific protein-probe complex and the position of free probe. Nuclear proteins were extracted from lung tissue of BNR treated with none (lane 1 and 2), OA (lane 3 and 4), OA plus F-4 (prophylaxis)(lane 5 and 6) and OA plus F8 (prophylaxis)(lane 7 and 8). 1 ng antibody against p50 was added for super-shift assay (lane 2, 4, 6 and 8).

FIG. 31 The correlation between cumulative inhalation units and FEV1.0 in F8.

### DETAILED DESCRIPTION OF THE PREFERRED EMBODIMENTS OF THE INVENTION

This invention involves two main areas of investigation:

A: Isolating the specific fractions F-4, and the compound F8.

B: Methods and processes to extract the above-mentioned fractions and a compound F8.

The above active fractions and active compounds can be used for inhibition of PAF induced rabbit platelete aggregation in vitro, and the future clinical application of them to reduce the bronchial hyperresponsiveness, histopathological changes and enhanced Th1 cytokines inhibiting Th2 cytokines and iNOS gene expression in BNR asthma model.

In terms of inhibition, PAF induced rabbit platelete aggregation is used as an index of PAF function inhibition. It was used as the first step in vitro screening method for identifying potential substances which are capable of inhibiting PAF function and may improve bronchial hyperresponsiveness in vivo.

An in vitro Screening System

Washed platelets were obtained from rabbits. In brief, rabbit blood was collected from the marginal ear vein into tubes containing one-sixth volume of acid-citrate-dextrose as anticoagulant. The blood was centrifuged at 200×g for 15 min at room temperature. The platelet-rich plasma was mixed with 1/40 volume of EDTA (final concentration 5 mM) and re-centrifuged at 1000×g for 12 min. The supernatant was discarded and the platelet pellet was suspended in modified Ca²⁺-free Tyrode's buffer (137 mM NaCl, 2.8 mM KCl, 2 mM MgCl₂, 0.33 mM NaH₂PO₄, 5 mM glucose, 10 mM HEPES) with 0.35% bovine serum albumin, heparin (50 unit/ml) and apyrase (1 unit/ml). Following incubation at 37° C. for 20 min, the washed platelet pellet was resuspended in Tyrode's buffer containing 1 mM Ca²⁺. The platelet number were counted by using a hemocytometer and adjusted to 3.5×10⁸ platelets/ml. To eliminate or minimize any possible effects of the solvent, the final concentration of vehicle dimethyl sulfoxide (DMSO) in the platelet suspension was fixed at 0.5%. Washed platelets (0.5 ml) were preincubated with 2.5 ┌l of the vehicle DMSO (0.5%) or Cordyceps sinensis active fraction F-4 (500 ┌g/ml) or active component F-8 (0.625 mM) for 2 min and then stimulated with 2.5 ┌l of platelet aggregation factor (PAF; 2 nM). Aggregation was measured by a turbidimetric method. The PACKS-4 aggregometer (Helena Laboratories, Beaumont, Tex., USA) was used to record platelet aggregation. Transmission of washed platelet suspension was assigned 0% aggregation while transmission through Tyrode's buffer was assigned 100% aggregation. The inhibitory activities of F-4 and F-8 on platelet aggregation induced by PAF were calculated according the equation:

$$Inhibition ( \% ) = \frac{{[ Aggregation \% ]}_{DMSO} - {[ Aggregation \% ]}_{Drug}}{{[ Aggregation \% ]}_{DMSO}} \times 100$$

An in vivo Screening System

The requirements for animal models include both specificity and the capacity for developing pulmonary histopathological lesions that are similar to those found in the corresponding human disorders. In view of the above considerations, three kinds of animal models were adopted for the experiments used in developing this invention. Two were invasive method and one was non-invasive method. The first and second invasive models were BNR and guinea pig asthma model induced by ovalbumin (OA). Twenty four either BNR (weight ranging from 250˜350 g) or guinea pig (380˜500 g) were divided into three groups. Each group consisted of 8 weight-matched male animals. Both group I and group II BNR were put into a closed chamber (30×30×16 cm plastic box) with two small holes, one serving as a gas inlet and the other as a gas outlet. Two mL of 1% OA was aerosolized by nebulizer and delivered continuously into the closed chamber with a gas delivery flow of 8 l/min. The animal was exposed to OA in the chamber for 10 minutes.

A second sensitization was performed 7 days later using the same procedure. Another 7 days later, a provocation test was performed. To prevent anaphylaxis and possible death, the animals were pretreated with pyrinamine (10 mg/kg) intraperitoneally 30 minutes before the test. For provocation, the BNR inhaled 3 mL of 4% aerosolized OA by nebulizer for 10 minutes in a closed chamber, using the same procedure as for sensitization. Group I BNR or guinea pig were given Cordyceps sinensis active fraction F-4 4 mg/kg intraperitoneally 30 minutes before OA inhalation provocation. Group II control animals were treated in the same way as group II, except that they were sensitized and challenged by breathing aerosolized saline instead of OA and without Cordyceps sinensis pretreatment.

In the Cordyceps sinensis treatment study, the protocol is similar to pretreatment except F-4 is administed after OA inhalation provocation.

For the lung function and acetylcholine provocation portion of the study, all animals were anesthetized with sodium pentobarbitone (50 mg/kg intraperitoneally). Jugular vein cannulation (PE-10 polyethylene tube filled with heparin, 1000 iu/mL in normal saline) and tracheostomy were performed. Lung function test was done 24 hours after the OA provocation test. The BNRs or pignea pigs were put in a body box (Model 6 Body Box, Buxco, Troy, N.Y., USA) in a supine position while the mice were put in a body box for mice (Anesthetized Body Box for Mice, Buxco, Troy, N.Y., USA). Gallamine triethiodide (4 mg/kg) was intravenously injected to induce paralysis and inhibit spontaneous breathing. A small animal ventilator set at a tidal volume of 6 mL/kg and a respiratory rate of 60 times/minute for the BNRs, 120 times/minute for the mice was used to ventilate the small animals. All animals were stable without spontaneous breathing 5 minutes after gallamine was given. Pulmonary function tests (PET) as described below were measured at baseline. Therefore, 25, 50, 75 and 100 μg/kg of acetylcholine (Ach) were given intravenously at 30 minute intervals. PETs were done 5 seconds after each dose of acetylcholine. Before each successive dose of Ach, the flow volume loop returned to baseline.

The airway opening pressure (PaO) was measured by a Gould pressure transducer at the tracheotomy. Respiratory flow was measured by a DP-45-14 differential pressure transducer. When tidal breathing was examined, 3-layer 325-mesh wire screen was used to measure the pressure difference. When a maximal forced expiratory maneuver (MFEM) was performed, a 6-layer 325-mesh wire screen was used to measure the pressure difference.

For the MFEM, the lungs were inflated to total lung capacity 9TLC, lung volume at PaO=35 cm H₂O) 3 times; the inflation was regulated by a solenoid valve. At peak volume during the third inflation, the inflation valve was shut off and another solenoid valve was opened immediately for deflation. The deflation valve was connected to a 20-liter container, with a pressure of −40 cm H₂O (subatmospheric). The negative pressure of 40 cm H₂O produced maximal expiratory flow (Vmax).

Changes in flow, volume, and PaO were recorded by a 7P1, 7P10 preamplifier (Grass Instrument Company, Quincy, Mass., USA) from the body box and the flow volume was stored in an oscilloscope (Hitachi Den Shi American Ltd, N.Y., USA). The parameters of the flow volume loop (FV 100p), including peak flow (the maximal flow rate of the FV 100p), MFEF 75% (the flow rate at 75% TLC), MFEF 50% (the flow rate at 50% TLC) and MFEF 25% (the flow rate at 25% TLC) were recorded.

TLC was defined as the gas volume in the lungs at airway pressures of +35 cm H₂O (TLC). The lungs were deflated to RV by connecting one port of the respiratory valve to a 4-liter reservoir at −10 cm H₂O=; the valves were then turned and the lungs inflated by a syringe to TLC.

Neon and CO concentrations were measured on a gas chromatograph for respiratory gases (model AGC 111, Carle Instruments, Fullerton, Calif., USA). At the same time, lung volumes (such as TLC and FRC) were measured using the gas (neon) dilution principle. TLC was measured three times and the average valve was reported.

After completion of the pulmonary function tests, BAL was performed using 10 mL normal saline twice (total 20 mL) for BNR or guinea pig. The BAL fluid was collected into plastic flasks containig 1,500 units of heparin and then strained through 1 layer of surgical gauze and centrifuged at 500 g for 5 min. The cell pellet was washed 3 times with sterile saline solution and resuspended in RPMI-1640. A small portion was taken for evaluation of cell number and viability, as assessed by trypan blue exclusion.

After BAL, each animal's chest was opened and the lungs were removed. The trachea and right lower lobe was fixed in 10% formaldehyde, dehydrated by different concentrations of alcohol, then embedded in paraffin, and cut into 4 μm thicknesses, stained with haematoxylin and eosin, and examined by light microscope.

Among pulmonary function change in OA induced bronchial hyperresponsiveness, cell components changes in BAL and histological change are similar to those found in human bronchial asthma i.e. obstructive lung function change, eosinophil and mononuclear cells infiltrations and bronchial epithelial slough.

The thirds was non-invasive model. For the determination of airway responsiveness in non-invasive method, airway responsiveness (AR) was measured in unrestrained BALBc mice by barometric plethysmography using whole body plethysmography (WBP) (Buxco, Troy, N.Y., USA). Before taking readings, the box was calibrated with a rapid injection of 1 ml of air into the main chamber to obtain the 1 mv signal from the WBP.

Inspiration and expiration were recorded by establishing start-inspiration and end-inspiration as the box pressure/time curve crossed the zero point. Start of an inspiration was determined by extrapolating from a straight line drawn from two levels of the rising inspiratory phase of the box pressure signal. Time of inspiration (TI) was defined as the time from the start of inspiration to the end of inspiration; time of expiration (TE) as the time from the end of inspiration to the start of the next inspiration. The maximum box pressure signal occurring during one breath in a negative or positive direction was defined as peak inspiratory pressure (PIP) or peak expiratory pressure (PEP), respectively. Recordings of every 10 breaths were extrapolated to define the respiratory rate in breaths per minute. The relaxation time (Tr) was defined as the time until a 36% of the total expiratory pressure signal (area under the box pressure signal in expiration) occurred. This served as a correlate to the time constant of the decay of the volume signal to 36% of the peak volume in passive expiration. Pause and Penh was defined and calculated by the following formulae:

Pause=(Te−Tr)/Tr

Penh (Enhanced Pause)=(PEP/PIP)* Pause

The mice were placed in the chamber, and baseline readings were taken and averaged for 3 minutes. Compressed air from a cylinder was passed through a regulatory set to deliver 20 psi. Thereafter, the compressed air was passed through a flow meter adjusted to deliver a gas flow of 8 l/min for 3 minutes and was then aerosolized through the inlet of the nebulizer. The output of the nebulizer was three mL of aerosolized PBS or methacholine in increasing concentrations (0.5, 1, 5, 25 mg/ml), which was delivered continuously into the closed chamber containing the mouse. Airway reactivity was expressed as an increase in concentration of Mch (PenhMch) compared with penh values after PBS challenge (Penh PBS).

An increase in Penh of 100% from baseline after methacholine was considered a positive challenge test. For quantification of the dose-response to methacholine, the results of the provocation test were expressed by a dose-response curve plotted on semilog paper. The linear ordinate represents the Penh values. The provocative concentration required to increase the Penh by 100% was then calculated from the curve and expressed as a dose of methacholine (PC100PenhMch).

Study Protocol

In the nonanesthetized condition, 24 hours after airway challenge by 3 mL 1% OA aerosol, pulmonary function testing (PET) was measured by WBP at baseline and after methacholine (Mch) inhalation challenge. Four hours after PET in a nonanesthetized condition, all animals were anesthetized with sodium pentobarbitone (50 mg/kg intraperitoneally). Jugular vein cannulation (PE-10 polyethylene tube filled with heaprin, 1000 iu/mL in normal saline) and tracheostomy were performed. The animals were put in a body box (anesthetized small animal body box, Buxco, USA) in a supine position. Gallamine triethiodide (4 mg/kg) was injected intravenously to induce paralysis and inhibit spontaneous breathing. A small animal ventilator set at a tidal volume 6 mL/kg and a respiratory rate of 120 times/minute was used to ventilate the animals by room air. All animals were stable without spontaneous breathing 5 minutes after gallamine was given. Pulmonary function tests, including flow volume loop, peak airway opening pressure (PaO), and gas flow to calculate total dynamic lung compliance, were examined at baseline. Thereafter, 25, 50, 75 and 100 μg/kg of acetylcholine were given intravenously at 30 minute intervals. Pulmonary function test were done 5 seconds after each dose of acetylcholine. Before each dose of acetylcholine the flow volume loop returned to baseline.

Pulmonary Function Tests

The PaO was measured by a Gould pressure transducer at the tracheostomy. PaO was defined as opening airway pressure during tidal breathing when a small animal ventilator was used (tidal volume 6 mL/kg, respiratory rate 120 times/minute). Respiratory flow was measured by a DP-45-14 differential pressure transducer.

For the maximal forced expiratory maneuver (MFEM), the lungs were inflated to total lung capacity (TLC, lung volume at PaO =35 cm H₂O) 3 times; the inflation was regulated by a solenoid valve. At peak volume during the third inflation, the inflation valve was shut off and the other solenoid valve was used immediately for deflation. The deflation valve was connected to a 20 liter container with a pressure of −40 cm H₂O (subatmospheric). The negative pressure produced maximal expiratory flow (Vmax). The changes in flow, volume and PaO were recorded by a 7P1, 7P10 preamplifier (Grass instrument Company, USA) from the body box and flow volume was stored in an oscilloscope (Hitachi Den Shi America Ltd, New York, USA). The parameters of the flow volume loop (FV loop), including peak flow (the maximal flow rate of the FV loop), MFEF75% (the flow rate at 75% TLC), MFEF50% (the flow rate at 50% TLC) and MFEF25% (the flow rate at 25% TLC) were recorded.

PD20MFEF50%Ach is the dose of acetylcholine required to produce a decrease in MFEF50% of 20% from baseline. PD20MFEF25%Ach is the dose required to produce a decrease in MFEF25% of 20% from baseline. PD50PaOAch is the dose required to produce an increase in PaO of 50% from baseline. PD50CdynAch is the dose required to produce a decrease in Cdyn of 50% from baseline. PD50RawAch is the dose required to produce an increase in Raw of 50% from baseline after acetylcholine was given.

Measurement of Lung Volumes

TLC and residual volume (RV) were defined as the gas volume in the lungs at airway pressures of +30 cm H₂O (TLC) and −10 cm H₂O (RV) respectively. The lungs were deflated to RV by connecting one port of the respiratory valve to a 4-liter reservoir at −10 cm H₂O; the valve was then turned, and the lungs were inflated from a syringe to TLC. The volume necessary to inflate the lungs from −10 to 30 cm H₂O was read from the syringe and was recorded as the vital capacity (VC). A specific measurement sequence was used throughout the study. VC was determined three times and then TLC was measured three times each. The average valve of the three measurements was reported.

Lung volumes (such as TLC and FRC) were also measured using the gas (Neon) dilution principle. Ne concentrations were measured on a gas chromatography for respiratory gases (model AGC 111, Carle Instruments, Fullerton, Calif., USA).

Bronchoalveolar Lavage (BAL)

After completion of the pulmonary function tests, BAL was performed using 1 mL normal saline twice (total 2 mL). The BAL fluid was collected into plastic flasks containing 1,500 units of heparin and then strained through 1 layer of surgical gauze and centrifuged at 500 g for 5 min. The cell pellet was washed 3 times with sterile saline solution and resuspended in RPMI-1640. A small portion was taken for evaluation of cell number and viability, as assessed by trypan blue exclusion.

Differential counts were obtained using a cytocentrifuge preparation (Cytospin; Shandon Southern Instruments, Sewickley, Pa.) stained with Liu's stain (modified May-Giemsa stain).

Histological Examination

After BAL, each animal's chest was opened and the lungs were removed. Histologic specimens (n=6 per each time point) were prepared. The trachea and each lobe were fixed in 10% formaldehyde, dehydrated by different concentrations of alcohol, then embedded in paraffin, and cut into 4 μm thicknesses; stained with haematosylin and eosin, and examined by light microscope for evaluation of the severity of inflammation. Each trachea and lung section was blindly assigned an inflammation score by two pathologists as follows; 1=rare or occasional inflammation cells scattered through the lung or trachea; 3=abundant inflammatory cells scattered through the lung or trachea; 2=inflammatory cells between the levels of 1 and 3. The total inflammation score for each animal was calculated as mean of the scores for 5 lung sections and trachea.

Data Analysis

The student's t-test and the ANOVA test were used for statistical analysis where appropriate. If the ANOVA test showed statistical significance, the Scheffe test was also done. All values were expressed as the mean±standard deviation, with significance accepted when p<0.05. Simple linear regression was used for correlation analysis.

The in vivo role of F-4 and F8 of Th1/Th2 cytokines by mesangial mRNA in lung tissue and nuclear factor (NF)κ B activity using an electromobility shift assay (EMSA)

Cytokine mRNA Analysis

Total RNA was extracted from lung tissue samples by a method described previously and RT-PCR was performed using a RT-PCR kit (Clontech, USA). The following T-cell related cytokines were evaluated by measuring their mRNA expression: IFNγ (Th-1 related) and IL-4, IL-5 and IL-10 (all Th-2 related) and inducible nitric oxide synthase (iNOS).

Preparation of cDNA and PCR Analysis

Briefly, 2 μg of RNA in a 25 μl volume was first primed with 2 μl oligo-(dT)₁₈ primer, 20 μM, at 70° C. for 2 min and then kept on ice for 5 min. after the reaction, a mixture containing the following: 8 μl of 5×reaction buffer; 2 μl, dNTP mix 10 μM (each); 1 μl recombinant Rnase inhibitor, 40 units/μ1; and 2 μl MMLV reverse transcriptase, 200 units/μ1, was added to synthesize cDNA. cDNA was amplified by 40 PCR cycles; each consisting of a denaturation step (94° C. for 1 min), an annealing step (65˜55° C. for 1 min, 0.5° C./cycle touch down, 55° C. for 19 cycles), and an extension step (72° C. for 1 min). During the last cycle the 72° C. step was extended to 5 min. The PCR product was analyzed on 2% agarose gel electrophoresis. The identity of each cytokine cDNA was determined by the size of the PCR product obtained by electrophoresis, and confirmed by DNA sequencing of the PCR products. The copy number was determined where the intensity of the input template RNA was equal to the intensity of the unknown sample. The band intensity values were normalized for their molecular weight, and the log of the ratio of the band intensities within each lane was plotted against the copy number of the template added per reaction. The quantities of target messanges were determined where the ratio of template and target hand intensities was equal to 1 and were analyzed by equations of the β-actin line.

Electromobility Shift Assay

The double-stranded NF-κ B consensus sequence 5′-AGTTGAGGGGACTTTCCCAGG-3′ was purchased from Promega (Madison, Mich., USA) and labeled with (³²P) using T4 kinase (Promega) and (³²P) ATP (Amersham Corporation, Arlington Heights, Ill., USA). The binding of nuclear protein to the radiolabeled oligonucleotide was performed. After 15 min at room temperature (generally 21˜23° C.), the binding mixture was applied to a 22-cm long 6% non-denaturing acrylamide gel in 0.5×Tris, borate, and ethylenediaminetetraacetic acid buffer and electrophoresed at 300V for about 2 h. The gel was subjected to radioautography using Kodak Biomax film with an intensifying screen.

In the next experiments, antibody against the p50, p52, p65, c-rel or Rel-B subunits (Santa-Cruz Biotechnology, Santa Cruz, Calif., USA) was added to the binding reaction mixture either 20 min or 14 h prior to addition of labeled oligonucleotide. Regardless of the preincubation time, electrophoresis was performed for 3 h to maximize the separation of proteins and achieve some degree of resolution between the migration of different homodimer and heterodimer complexes that bound to the labeled oligonucleotide. As a control, and antibody to the transcription factor c-fos (SC-52-G; Santa-Cruz Biotechnology) was added to the binding reaction mixture to demonstrate the specificity of binding of the NF-κ B subunit antibodies.

Results

Subject Animals and Study Protocol

Study I: BNR or Guinea Pig Sensitzed and Challenged by OA with or without F-4

Results

Subject Animals and Study Protocol

Study I: BNR or Guinea Pig Sensitzed and Challenged by OA with or without F-4 Pretreatment

Study Protocol

Twenty four BNR (weight ranging from 250˜350 g) or guinea pigs (weight ranging from 380˜500 mg) were divided into three groups. Each group consisted of 8 weight-matched male animals. Aerosol sensitization and challenge with OA were performed on both group I and group II.

Both group I and group II BNR or guinea pigs were put into a closed chamber (30×30×16 cm plastic box) with 2 small holes, 1 serving as a gas inlet and the other as a gas outlet. Two mL of 1% OA was aerosolized by nebulizer and delivered continuously into the closed chamber with a gas delivery flow of 8 l/min. The animal was exposed to OA in the chamber for 10 minutes.

A 2nd sensitization was performed 7 days later using the same procedure. Another 7 days later, a provocation test was performed. To prevent anaphylaxis and possible death, the animals were pretreated with pyrinamine (10 mg/kg) intraperitoneally 30 minutes before the test. For provocation, the BNR or guinea pigs inhaled 3 mL of 4% aerosolized OA by nebulizer for 10 minutes in a closed chamber, using the same procedure as for sensitization. Group I BNR or guinea pigs were given F-4 4 mg/Kg intraperitoneally 30 minutes before OA inhalation provocation. In contrast, group II BNR were only given vehicle intraperitoneally. Group III control animals were treated in the same way as group II, except that they were sensitized and challenged by breathing aerosolized saline instead of OA and without F-4 pretreatment.

Results

Pulmonary Function Test Data

The mean data at baseline of peak flow, MFEF 75%, MFEF 50% and MFEF 25% of the flow volume loop for all 3 groups of BNR was shown in Table 1 and of guinea pigs was shown in Table 2. There was no difference in peak flow, MFEF 75%, PaO, Cdyn and TLC among these 3 groups, but MFEF 50%TLC and MFEF 25%TLC were lower in group II when compared with group I and III (p<0.05) (Table 1 and Table 2).

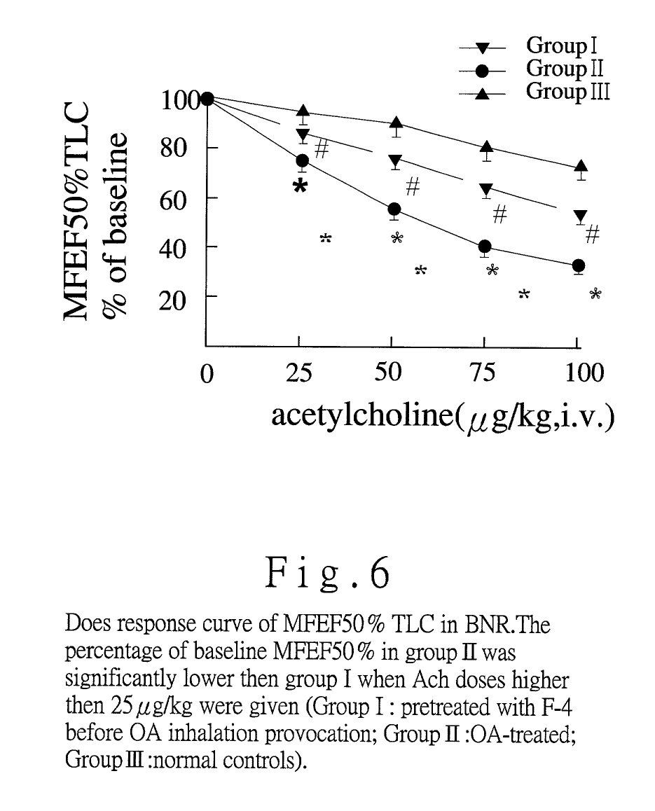

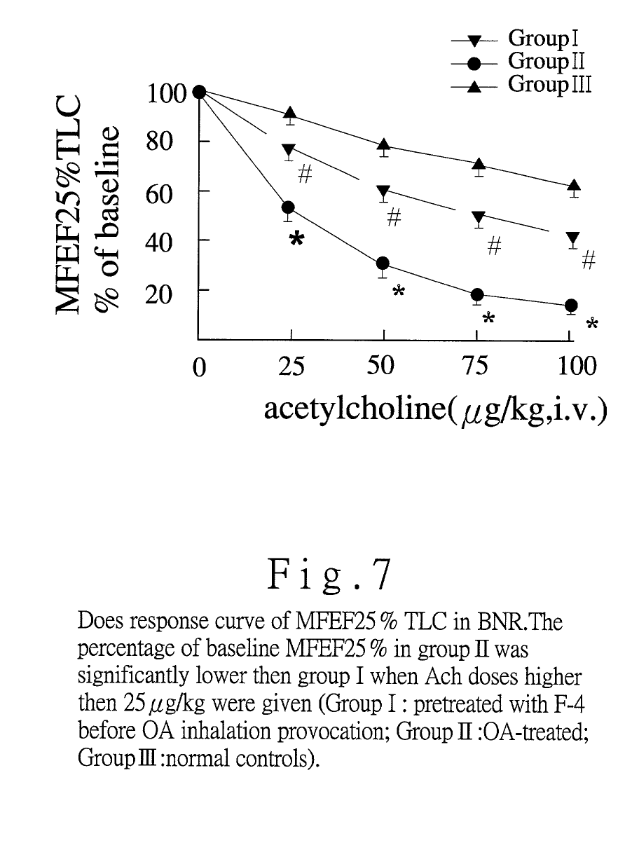

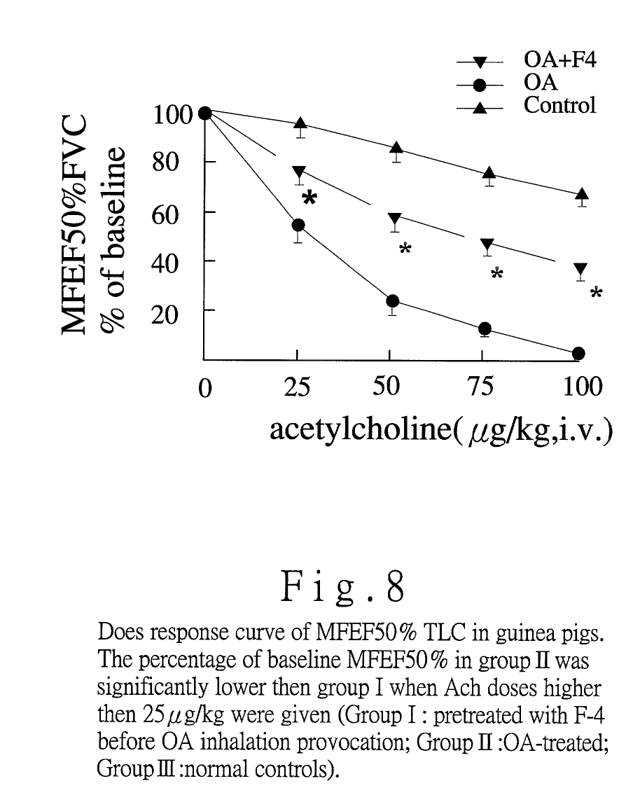

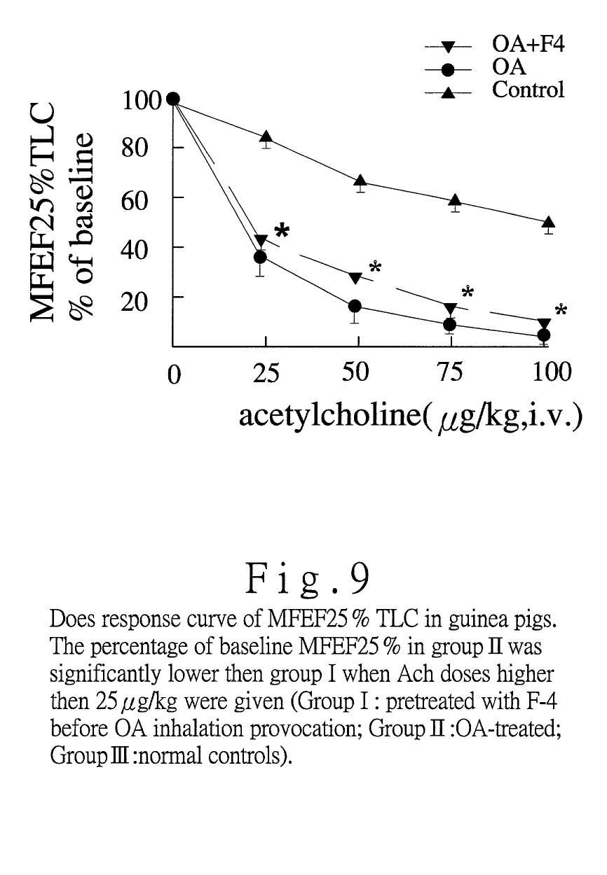

The percent change in MFEF 50%TLC and MFEF 25%TLC of either BNR (FIG. 6, FIG. 7) or guinea pigs (FIG. 8, FIG. 9) were higher in group II than in the other 2 groups at doses of Ach higher than 25 μg/kg. PD20 Ach, the dose of Ach producing a 20% drop in each PFT parameter, was significantly (p<0.001) lower in group II than in the other 2 groups for each of the following parameters: MFEF 50%, and MFEF 25% (p<0.05).

Bronchoalveolar Lavage

Group II had higher total cell counts than the other 2 groups. The percentage and absolute counts of eosinophils and lymphocytes in group II was higher than in the other 2 groups. In contrast, the percentage of macrophages was decreased in group II more than other 2 groups (Table 3 and Table 4).

Study II: Mice Sensitized and Challenged by OA with or without F-4 Pretreatment Protocol

Twenty four BALBc mice (weight ranging from 27˜33 g) were divided into 3 groups of 8 weight-matched male animals each. The mean weight of group I was 30±2 g, of group II was 29±2 g, and of group III was 31±2 g. Aerosol sensitization and challenge with OA were performed on both group I and group II mice.

Both group I and group II mice were sensitized by intraperitoneal injection of 20 μg OA (Sigma, St. Louis, Mo.) emulsified in 2 mg aluminum hydroxide (Alum inject; Pierce Chemical, Rockford, Ill.) in a total volume of 100 μl on day 1 and 14. An airway challenge of OA (1% in PBS) for 20 min was given on days 28, 29, and 30 by ultrasonic nebulization and assessed on day 31 by MFEM for airway reactivity. Group I mice were given F-4 4 mg/Kg intraperitoneally 30 minutes before OA inhalation provocation. In contrast, group II mice were only given vehicle intraperitoneally. Group I control animals were treated in the same way as group II, except that they were sensitized and challenged by breathing aerosolized saline instead of OA and without F-4 pretreatment.

Results

Pulmonary Function Test Data

The mean data at baseline of peak flow, MFEF 75%, MFEF 50% and MFEF 25% of the flow volume loop for all 3 groups is shown in Table 4. There was no difference in peak flow, MFEF 75%, PaO and TLC among these 3 groups, but MFEF 50%TLC and MFEF 25%TLC and Cdyn were lower in group II when compared with group I and III (p<0.05) (Table 5).

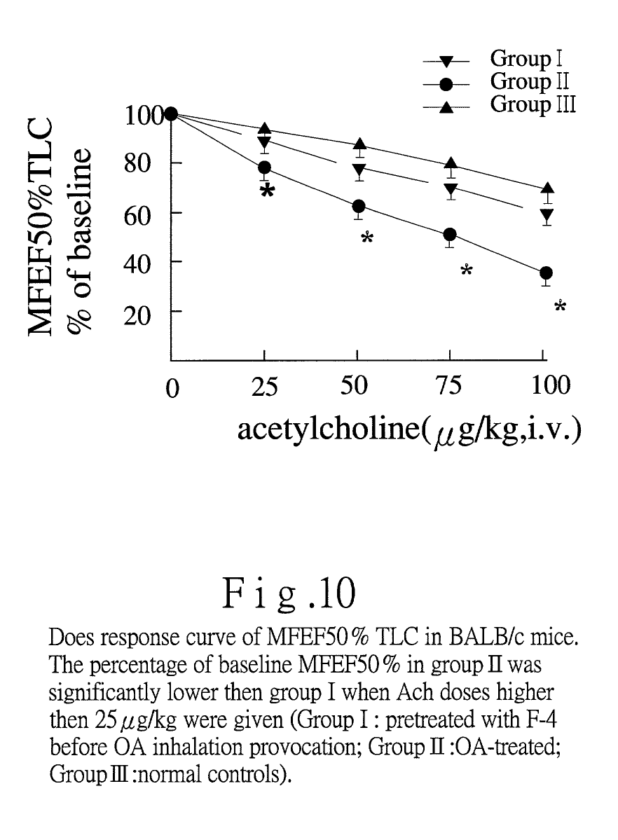

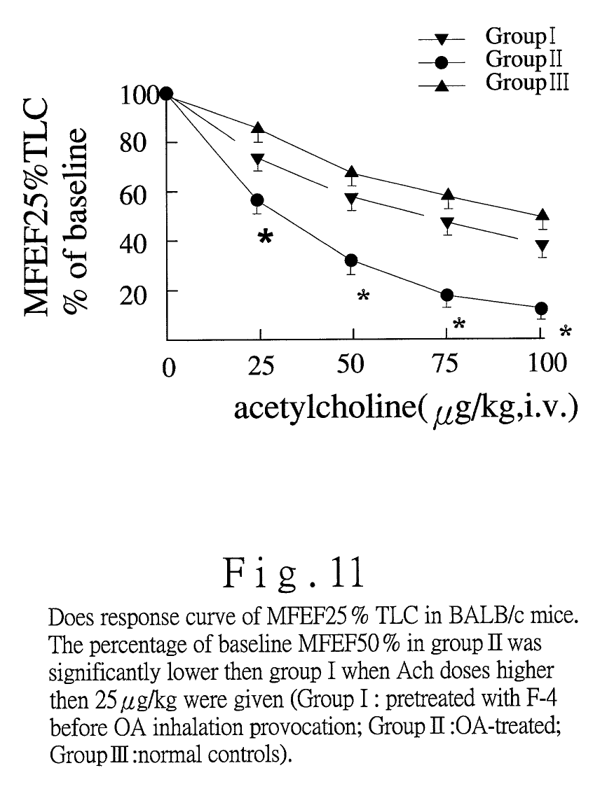

The percent change in MFEF 50%TLC and MFEF 25%TLC (FIG. 10, FIG. 11) were higher in group II than in the other 2 groups at doses of Ach higher than 25 μg/kg. PD20 Ach, the dose of Ach producing a 20% drop in each PFT parameter, was significantly (p<0.001) lower in group II than in the other 2 groups for each of the following parameters: MFEF 50%, and MFEF 25% (p<0.05).

Bronchoalveolar Lavage

Group II had higher total cell counts than other 2 groups. The percentage and absolute counts of eosinophils and lymphocytes in group II was higher than in the other 2 groups. In contrast, the percentage of macrophages was decreased in group II than in the other 2 groups (Table 6).

Study III: BNR and Guinea Pig Sensitized and Challenged by OA with or without F4 Treatment

Pulmonary Function Test Data

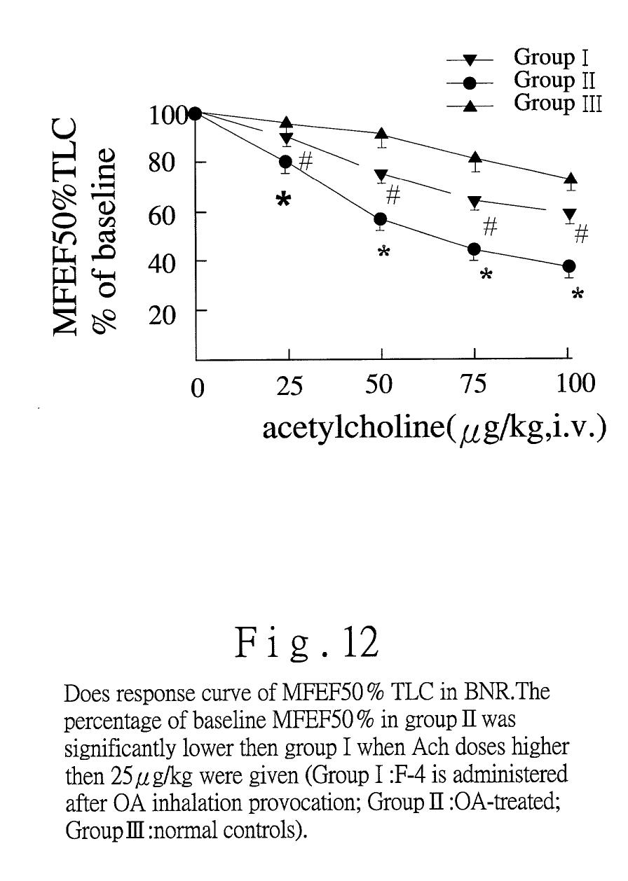

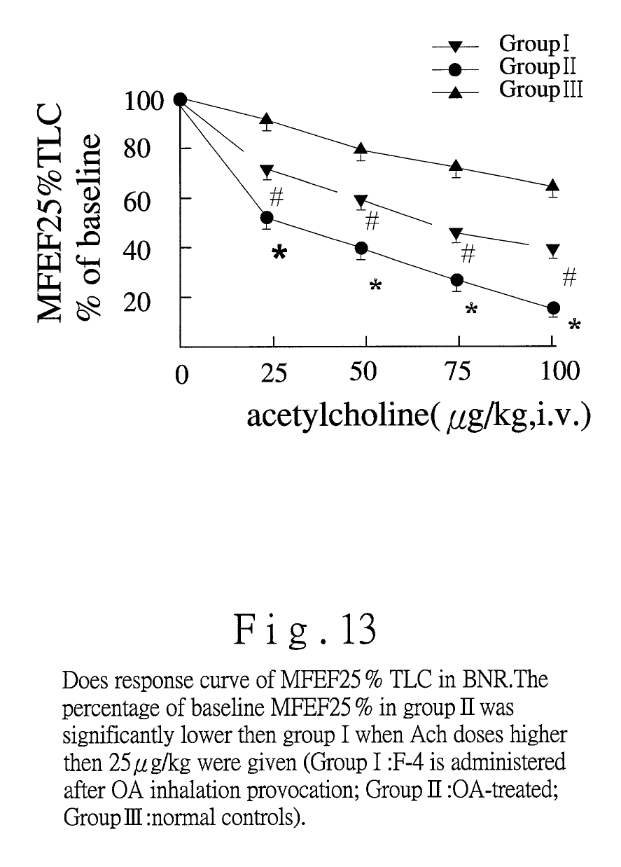

The percent change in MFFEF 50%TLC and MFEF 25%TLC of either BNR (FIG. 12, FIG. 13) or guinea pigs (Table 7) were higher in group II than in the other 2 groups at doses of Ach higher than 25 μg/kg. PD20 Ach, the dose of Ach producing a 20% drop in each PFT parameter, was significantly (p<0.001) lower in group II than in the other 2 groups for each of the following parameters: MFEF 50%, and MFEF 25% (p<0.05).

Bronchoalveolar Lavage

Group II had higher total cell counts than the other 2 groups. The percentage and absolute counts of eosinophils and lymphocytes in group II was higher than in the other 2 groups. In contrast, the percentage of macrophages was decreased in group II more than other 2 groups.

Study IV: Mice Sensitized and Challenged by OA with or without F4 Treatment

Pulmonary Function Test Data

The mean data at baseline of peak flow, MFEF 75%, MFEF 50% and MFEF 25% of the flow volume loop for all-3 groups is shown in Table 8. There was no difference in peak flow, MFEF 75%, PaO and TLC among these 3 groups, but MFEF 50%TLC and MFEF 25%TLC and Cdyn were lower in group II when compared with group I and III (p<0.05) (Table 8).

Bronchoalveolar Lavage

Group II had higher total cell counts than other 2 groups. The percentage and absolute counts of eosinophils and lymphocytes in group II was higher than in the other 2 groups. In contrast, the percentage of macrophages was decreased in group II than in the other 2 groups.

Study V: BNR Sensitzed and Challenged by OA with or without F8 Pretreatment

Study Protocol

Twenty four BNR (weight ranging from 250˜350 g) were divided into three groups. Each group consisted of 8 weight-matched male animals. Aerosol sensitization and challenge with OA were performed on both group I and group II.

Both group I and group II BNR were put into a closed chamber (30×30×16 cm plastic box) with 2 small holes, 1 serving as a gas inlet and the other as a gas outlet. Two mL of 1% OA was aerosolized by nebulizer and delivered continuously into the closed chamber with a gas delivery flow of 8 l/min. The animal was exposed to OA in the chamber for 10 minutes.

A 2nd sensitization was performed 7 days later using the same procedure. Another 7 days later, a provocation test was performed. To prevent anaphylaxis and possible death, the animals were pretreated with pyrinamine (10 mg/kg) intraperitoneally 30 minutes before the test. For provocation, the BNR inhaled 3 mL of 4% aerosolized OA by nebulizer for 10 minutes in a closed chamber, using the same procedure as for sensitization. Group I BNR were given F8 4 mg/Kg intraperitoneally 30 minutes before OA inhalation provocation. In contrast, group II BNR were only given vehicle intraperitoneally. Group III control animals were treated in the same way as group II, except that they were sensitized and challenged by breathing aerosolized saline instead of OA and without F8 pretreatment.

Results

Pulmonary Function Test Data

The mean data at baseline of peak flow, MFEF 75%, MFEF 50% and MFEF 25% of the flow volume loop for all 3 groups of BNR was shown in Table 7. There was no difference in peak flow, MFEF 75%, PaO, Cdyn and TLC among these 3 groups, but MFEF 50%TLC and MFEF 25%TLC were lower in group II when compared with group I and III (p<0.05) (Table 7).

Bronchoalveolar Lavage

Group II had higher total cell counts than the other 2 groups. The percentage and absolute counts of eosinophils and lymphocytes in group II was higher than in the other 2 groups. In contrast, the percentage of macrophages was decreased in group II more than other 2 groups (Table 8).

Study VI: Mice Sensitized and Challenged by OA with or without F8 Pretreatment Protocol

Twenty four BALBc mice (weight ranging from 27˜33 g) were divided into 3 groups of 8 weight-matched male animals each. The mean weight of group I was 30±2 g, of group II was 29±2 g, and of group III was 31±2 g. Aerosol sensitization and challenge with OA were performed on both group I and group II mice.

Both group I and group II mice were sensitized by intraperitoneal injection of 20 μg OA (Sigma, St. Louis, Mo.) emulsified in 2 mg aluminum hydroxide (Alum inject; Pierce Chemical, Rockford, Ill.) in a total volume of 100 l on day 1 and 14. An airway challenge of OA (1% in PBS) for 20 min was given on days 28, 29, and 30 by ultrasonic nebulization and assessed on day 31 by MFEM for airway reactivity. Group I mice were given F8 4 mg/Kg intraperitoneally 30 minutes before OA inhalation provocation. In contrast, group II mice were only given vehicle intraperitoneally. Group I control animals were treated in the same way as group II, except that they were sensitized and challenged by breathing aerosolized saline instead of OA and without F8 pretreatment.

Results

Pulmonary Function Test Data

The mean data at baseline of peak flow, MFEF 75%, MFEF 50% and MFEF 25% of the flow volume loop for all 3 groups is shown in Table 9. There was no difference in peak flow, MFEF 75%, PaO and TLC among these 3 groups, but MFEF 50%TLC and Cdyn were lower in group II when compared with group I and III (p<0.05) (Table 9).

Bronchoalveolar Lavage

Group II had higher total cell counts than other 2 groups. The percentage and absolute counts of eosinophils and lymphocytes in group II was higher than in the other 2 groups. In contrast, the percentage of macrophages was decreased in group II than in the other 2 groups (Table 10).

Study VII: BNR and Guinea Pig Sensitized and Challenged by OA with or without F8 Treatment

Pulmonary Function Test Data

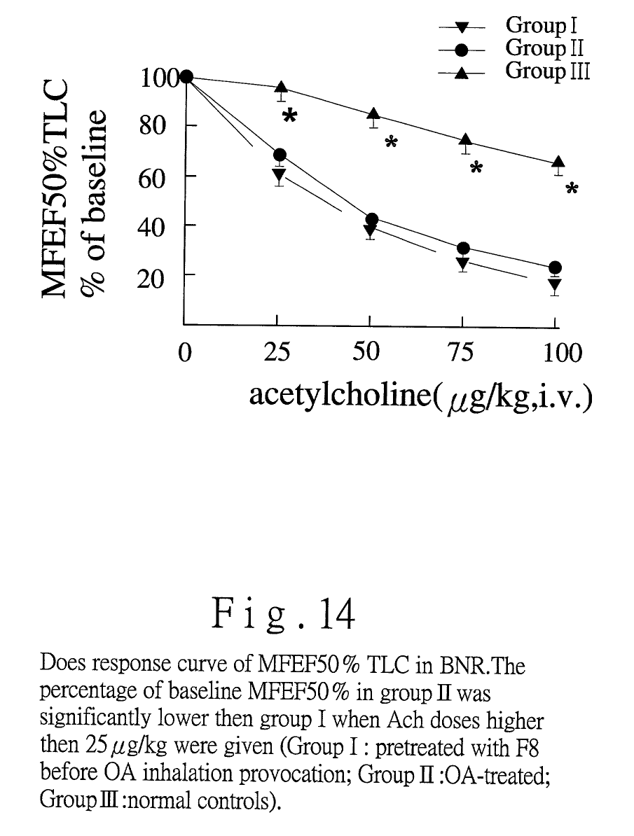

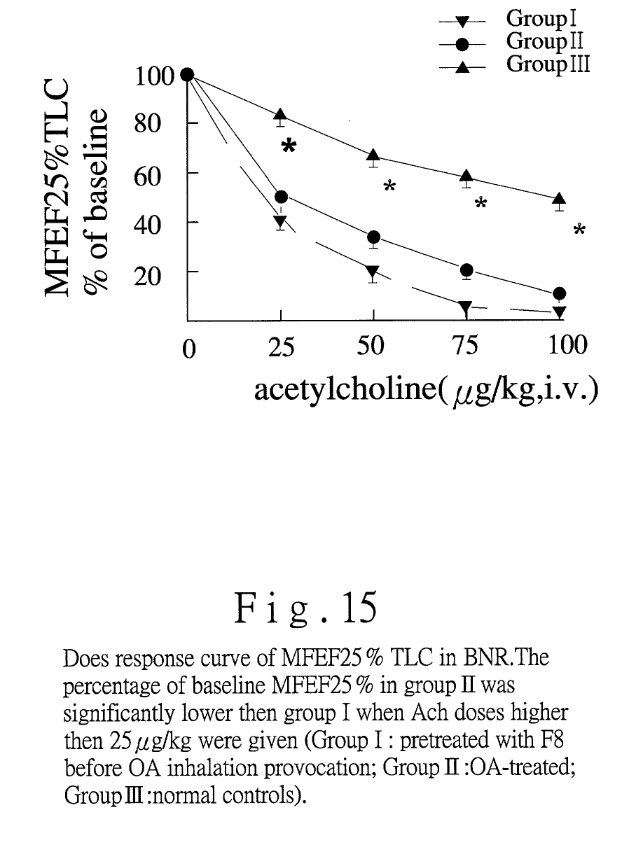

The percent change in MFEF 50%TLC and MFEF 25%TLC of either BNR (FIG. 14, FIG. 15) were higher in group II than in the other 2 groups at doses of Ach higher than 25 μg/kg. PD20 Ach, the dose of Ach producing a 20% drop in each PFT parameter, was significantly (p<0.001) lower in group II than in the other 2 groups for each of the following parameters: MFEF 50%, and MFEF 25% (p<0.05).

Bronchoalveolar Lavage

Group II had higher total cell counts than the other 2 groups. The percentage and absolute counts of eosinophils and lymphocytes in group II was higher than in the other 2 groups. In contrast, the percentage of macrophages was decreased in group II more than other 2 groups.

Study VIII: Mice Sensitized and Challenged by OA with or without F8 Treatment

Pulmonary Function Test Data

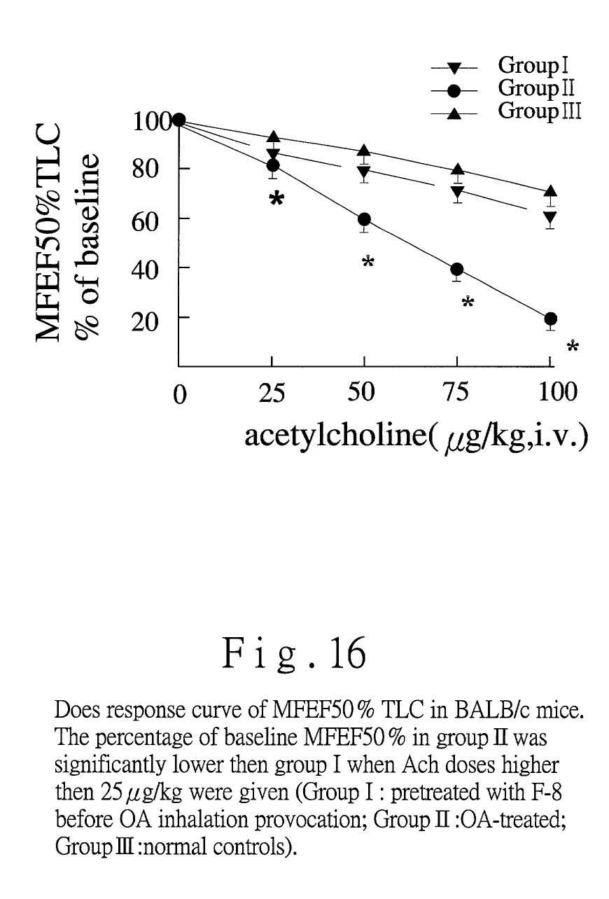

There was no difference in peak flow, MFEF 75%, PaO and TLC among these 3 groups, but MFEF 50%TLC and Cdyn were lower in group II when compared with group I and III (p<0.05) (FIG. 16).

Bronchoalveolar Lavage

Group II had higher total cell counts than other 2 groups. The percentage and absolute counts of eosinophils and lymphocytes in group II was higher than in the other 2 groups. In contrast, the percentage of macrophages was decreased in group II than in the other 2 groups.

Cytokine mRNA profile using RT-PCR (reverse-transcriptase polymerase chain reaction)

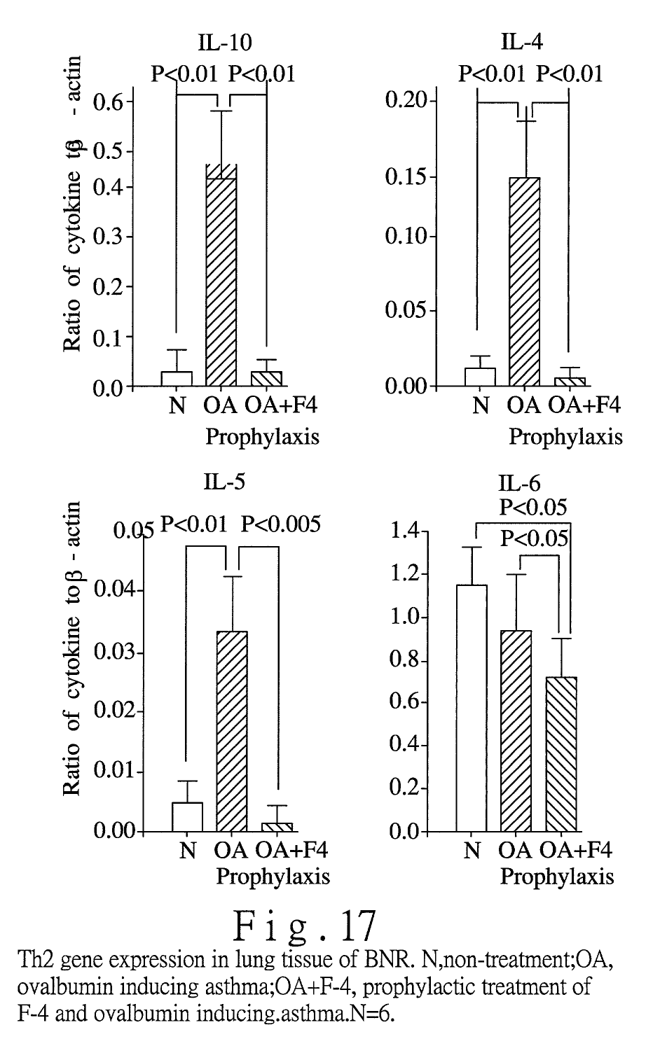

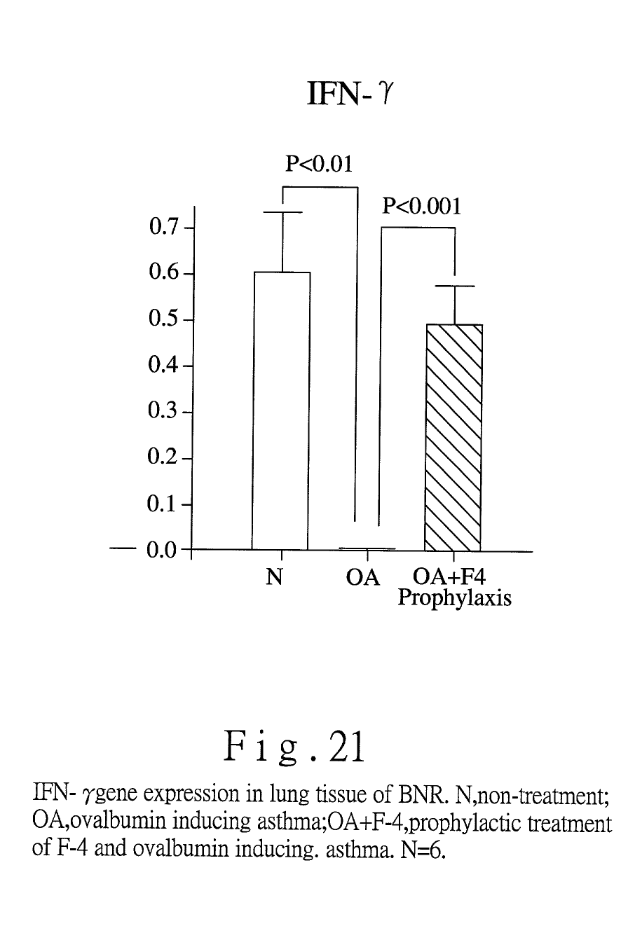

FIG. 17, 18, 19 & 20 demonstrated 1 example that group II had increased IL-4, IL-10 and iNOS mRNA expression compared to other 2 groups. FIG. 21, 22, 23, 24 demonstrated another example that group I and III increased IFN-γ mRNA expression than group II.

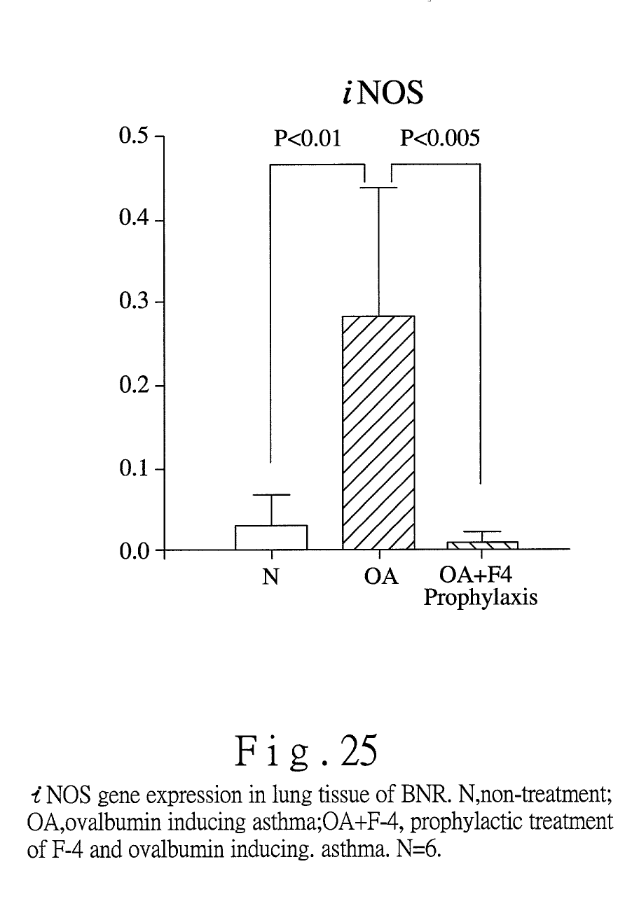

The ratio of IL-4, IL-5, IL-10 mRNA levels to β-actin in group II was significantly higher than in group II controls, as measured by densitometry. The iNOS β-actin ratio was significantly increased in group II animals (FIG. 25, 26, 27 & 28).

Correlation between BHR and Eosinophils with Cytokine mRNA Expression

The PD20 MFEF50% correlated negatively with IL-4 and IL-5 mRNA levels but positively with IFN-γ mRNA. There was also a positive correlation between the BAL eosinophil count and IL-4 and IL-5 mRNA but a negative correlation with IFN-γ mRNA.

Electromobility Shift Assay

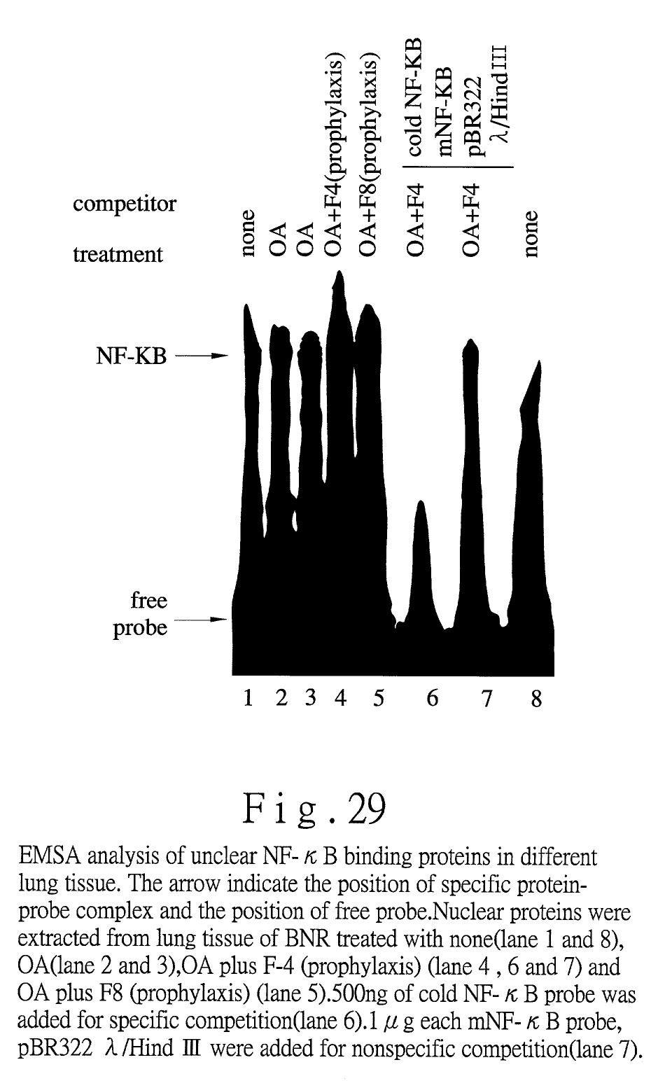

Nuclear extracts obtained from the lung tissue were subjected to EMSA using a [³²P]-labeled oligonucleotide representing the NF-K B consensus sequence (FIG. 29, 30). The excess unbound oligonucleotide probe migrated near the dye front on the autoradiograph replicas of the gels. The nuclear extracts bound labeled oligonucleotide and retarded their migration. There was a 10-fold excess of nuclear extracts obtained from the lung tissue of group I rats as compared with group II controls (FIG. 29), indicating that NF-κB activity was elevated in rat lung after OA sensitization and provocation.

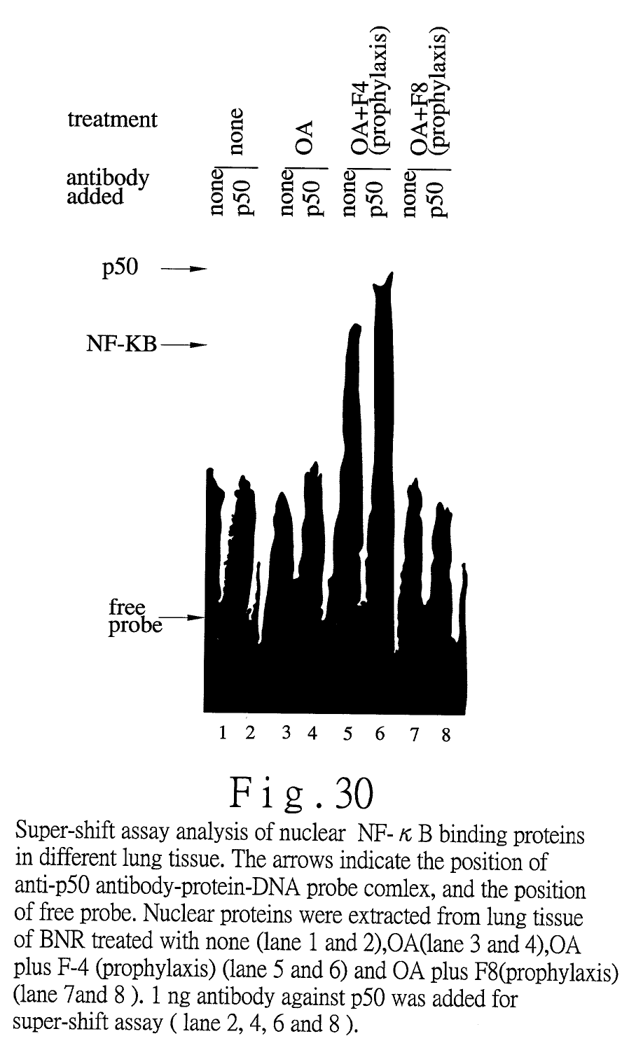

As mentioned above, the NF-κ B transcription factors comprise a family of protein that bind to DNA as a dimmer. Antibodies to individual proteins may be used in the binding assays to supershift or to deplete homodimeric or heterodimeric complexes that bind the radiolabeled oligopeptides. When the extract was preincubated with individual antibodies that cause a supershift (FIG. 30) for p50, or deplete binding for p65, well-defined bands of oligonucleotide binding remained. When the extract was preincubated with p50 antibody, the supershift was presence in the group I rats' lung tissue (FIG. 30) and absent in the controls' lung tissue (FIG. 30). When an antibody to p65 was used, there was no additional change in binding seen with antibody to the p65 subunit in either group. This results may indicate the extract was processed with an antibody to the p50 subunit of NF-κ B.

Histology of Lung Tissue

The airway and lung tissue of group I mice demonstrated a severe inflammatory reaction, characterized by hyperemia, interstitial edema and inflammatory cell infiltration. There was also evidence of airway epithelial cell desquamation. These changes were not seen in group II normal controls.

Correlation of Changes in MFEF with other Parameters and with BAL Eosinophile Count

There was a correlation between PD20MFEF50%Ach and PD20MFEF25%Ach with PD50PaOAch, PD50CdynAch, PD50RawAch (Table 4). There was also a positive correlation between the PD20MFEF50%Ach and PD20MFEF25%Ach with the eosinophil count in the BAL fluid.

In vitro Anti-PAF Activity

The effects of F-4 and F-8 on platelet aggregation induced by PAF are shown in Table 12. Cordyceps sinensis active fraction F-4 and active component F-8 induced a inhibition of aggregation of washed platelets induced by PAF (2 nM). At 500 μg/ml of FA, inhibited 83.8±9. 1% of PAF induced platelet aggregation. Incubation of washed rabbit platelet suspension with 0.625 mM F-8, PAF-induced aggregation was inhibited to 97±2.6%.

Toxicity

In ICR mice the LD₅₀ of Cordyceps sinensis was 21.7±2.6 g/kg for injection into the abdominal cavity, and 24.5+2.2 g/kg for injection into the tail vein. In terms of oral administration, the maximal tolerance dose is 252.5˜300 g/kg, a result which shows that irrespective of whether dosage is achieved by means of injection into the abdominal cavity or tail or by gastric implantation, this substance has a very low level of toxicity. The methodology used to carry out acute toxicity testing for this invention was as follows: ICR mice that had been fed on a normal diet with the above active compound included to constitute a 2% ratio were killed after 7 days in order to ascertain whether there was any evidence of toxicity. These results show that Cordyceps sinensis has a broad range of pharmacology actions and no acute toxicity (Table 13).

The methods used to obtain these fractions and compound F8 are detailed below:

Item One:

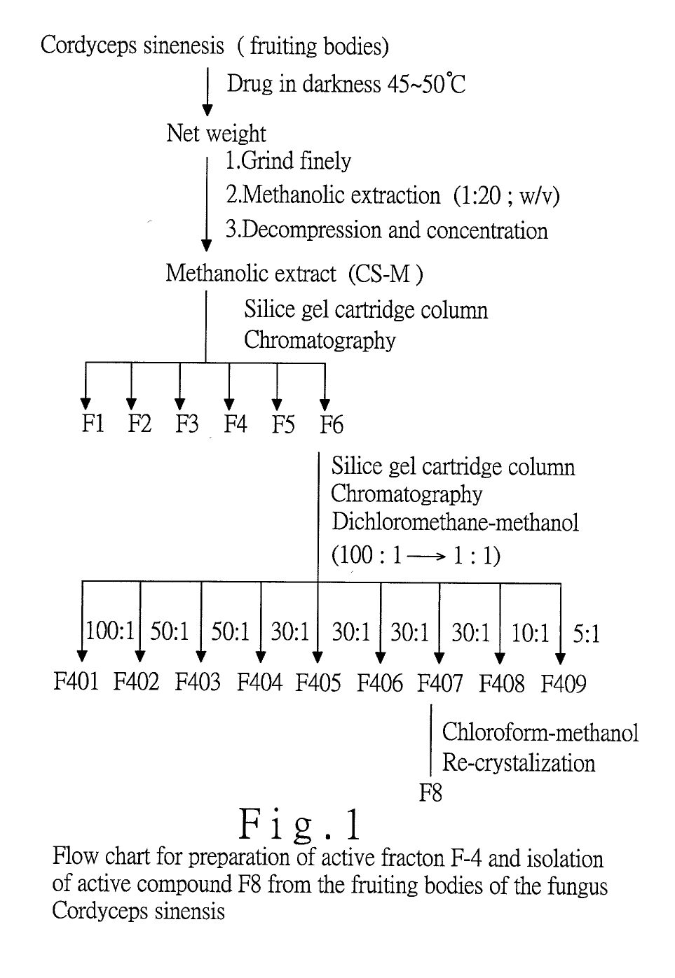

As shown in FIG. 1, this invention provides a method for obtaining fractions and a pure compound F8 from the stroma of Cordyceps sinensis. First, the sample is either air dried or in an oven (35˜60° C.). Cordyceps sinensis has a very high moisture content in its crude form, so drying is necessary to minimize the amount of polar substances that are drawn out in the extraction processes, as these would affect the results of silica gel column chromatographic purification. Next, the dried product is ground in a grinder or miller to increase the efficiency of extraction.

The polarity range of the active compound in Cordyceps sinensis (in terms of inhibiting PAF induced rabbit platelet aggregation and improving pulmonary function, histopathological changes and Th1 cytokines gene expression as described herein), is relatively low, so these substances can be effectively extracted by using methanol (or other low-carbon alcohol), acetone, diethyl ether, ethyl acetate, chloroform, or methylene chloride. However, considering the advantages of obtaining a high return of desired fractions and compound F8 with minimal extraction of polar contaminants, methanol and ethyl acetate are the most suitable choices. Methanol extraction was used as an example and the procedure is depicted in FIG. 1.

The chromatographic methods used are depicted in FIG. 1. The stroma of Cordyceps sinensis is either air dried or in an oven (45˜50° C.) for 2 days. Stroma sample is drawn out after dryness and incubated with 10×volume methanol for 48 hours, then the methanol is filtered and concentrated with a rotary vacuum concentrator, the solution is removed, the concentrated methanol is absorbed in adequate amount of silica gel 60, silica gel column chromatography is performed, different polarities of elution solutions are made by mixing various composition of n-hexane, ethyl acetate and methanol, finally six fractions (F1-F6) are separated by silica gel column chromatography. The flow chart is shown in FIG. 1. Fraction F4 has the most obvious biological activity of inhibiting rabbit platelets aggregation induced by PAF, the activity of active fraction persisting at least 3 months under 4° C.

In order to obtain the active compound from active fractions, F4 fraction is separated by silicon gel chromatography as follows (FIG. 1):

F4 is dissolved in small amount of methanol-chloroform, and separated by silicon (70-230 mesh) column (5×40 cm) chromatography; elution buffer is run in the order of methylene chloride-methanol (100:1→1:1), with collected elution solution in the composition of 10:1 (v/v). This solution has ability to inhibit rabbit platelets aggregation induced by PAF. After concentration and re-crystallization, active compound F8 can be obtained.

The method of culturing mycelia and confirming their presence is outlined below: culture a strain of Cordyceps sinensis (VGH-CS) in a liquid medium containing the following constituents:

| | |
|---|---|
| Glucose | 2% |
| Peptone | 0.5% |
| Malt extract | 2% |
| Potato-dextrose broth | 24 g/L |

Leave the culture at 26±1.0° C. for 30 days, then collect the mycelia and dry at 45 to 50° C. Grind the resulting mycelial products and place them in methanol at a ratio of 1:20 (dry weight/volume) for an extraction period of 24 hours. Concentrate the resulting crude extract. Carry out reversed-phase high performance liquid chromatographic analysis to ensure it contains the active compound F8.

In summary, item 1 covers both liquid cultured and semicultivated Cordyceps sinensis in a liquid-phase medium and methods for assaying the above-mentioned fractions and compound F8.

Item Two: Specific

A: Specific fractions: This term refers to those fractions that are obtained during the entire isolation process and in each chromatographic cycle, from methanol extraction to final purification of compound F8, and which demonstrate the strongest activity in vitro and improvement of bronchial hyperresponsiveness in vivo.

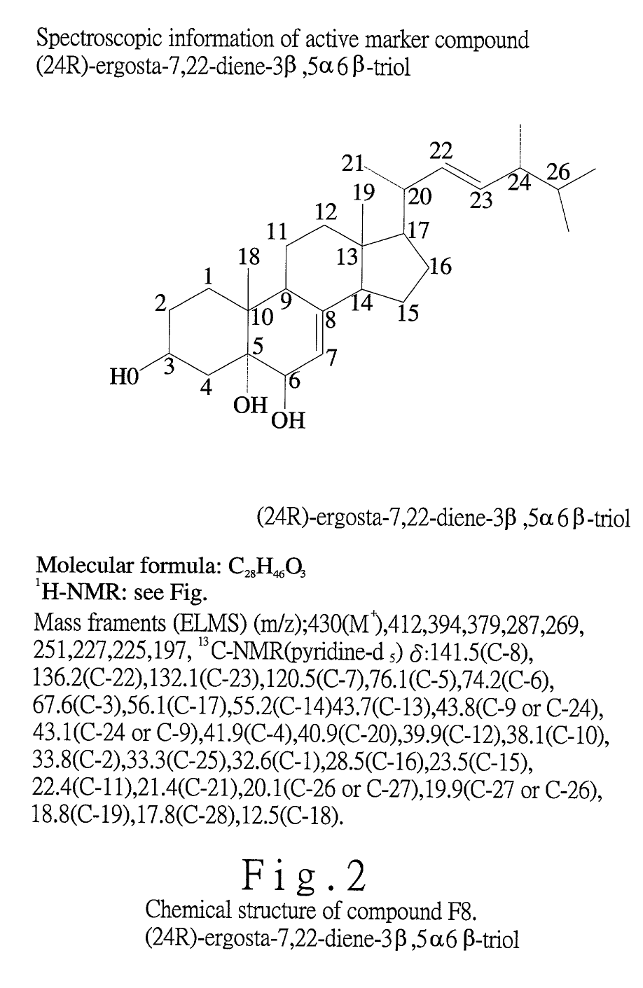

B: Compound F8: This refers to F8 (for spectroscopic and structural data, see FIG. 2a, 2b, and 3). The molecular formula of compound F8 is C₂₈H₄₆O₃. To confirm the potential applicability of the above fractions and to check for obvious toxicity or mutagenicity, Ames test and acute toxicity test were conducted on white mice (ICR-mice) using the F4 fraction or compound F8. The results showed no obvious evidence of toxicity or mutagenic properties.

Item Three: Pharmacological Effect in vivo

In vivo methods were adopted for the invention as discussed in the patient application. One of these utilizes in vivo improving of pulmonary function in bronchial asthma late phase response induced by OA in BNR models. The experimental animals BNR are divided with control group and experimental group, both of which are sensitized twice. The method takes 2 ml 1%OA solution to container of neubenlizer, neubenlized with 8 l/min flow rate, prescribed by inhalation to animals. The second sensitization is performed 7 days later, the procedure as described above. The examinations of pulmonary function are performed 7 days later after sensitization twice. Two days before examinations of pulmonary function, challenge with high dose OA is performed (3 ml 4% OA neubenlized). Intraperitoneal injection of pyrinamide (10 ng/kg) is administrated 30 minutes prior to challenge to avoid acute response and death. Acetylcholine provocation test is performed to BNR at 36 hours after challenge, pulmonary function is measured before and after (within 5 seconds) acetylcholine provocation test. In the late phase response of asthma in animals provocated with acetylcholine, there are obvious decreases of pulmonary functions (including lung vital capacity decreases, residual pulmonary volume increases, total lung capacity forced expiratory flow rate decreases, forced expiratory volume decreases), obvious features of obstructive lung change. In pathological examination, lavage amounts of monocyte and eosinophil infiltration and bronchial epithelium slough are found. The immunological change after prophylactic or therapeutic treatment is as follows:

(A) The Prophylactic Treatment with F4 or F8

In order to understand whether the extract of Cordyceps sinensis has prophylactic effect on asthma, the experiment is designed as inhalation with normal saline in OA group for control and inhalation with 3 ml F8 solution in prophylactic group before sensitization and challenge with OA.

(B) The Therapeutic Treatment with F4 or F8

After experimental animals are challenged, F4 or F8 is administrated intraperitoneally to experimental group and normal saline is injected to OA group for control.

The second in vivo method untilizes enhancing Th1 cytokines suppressing Th2 cytokines and expressing iNOS mRNA.

After treatment of the above animal models, the experimental animals are sacrified 2 days after challenge, lungs are removed and kept at low temperature in ice, RNA and nuclear protein are extracted and RT-PCR is performed.

The above results reveal that the active fractions F4 or active compound F8 of Cordyceps sinensis has effect, which is detected by the improvement of pulmonary function, pathological presentation and immunological change, to improve experimental asthma response either prescribed prophylactically (inhalation with 0.5 mg and 3 mg CS-F4 or F8 solution at the same time of second sensitization and challenge by OA) or prescribed therapeutically (intraperitoneal administration with F-4 or F8 after challenge).

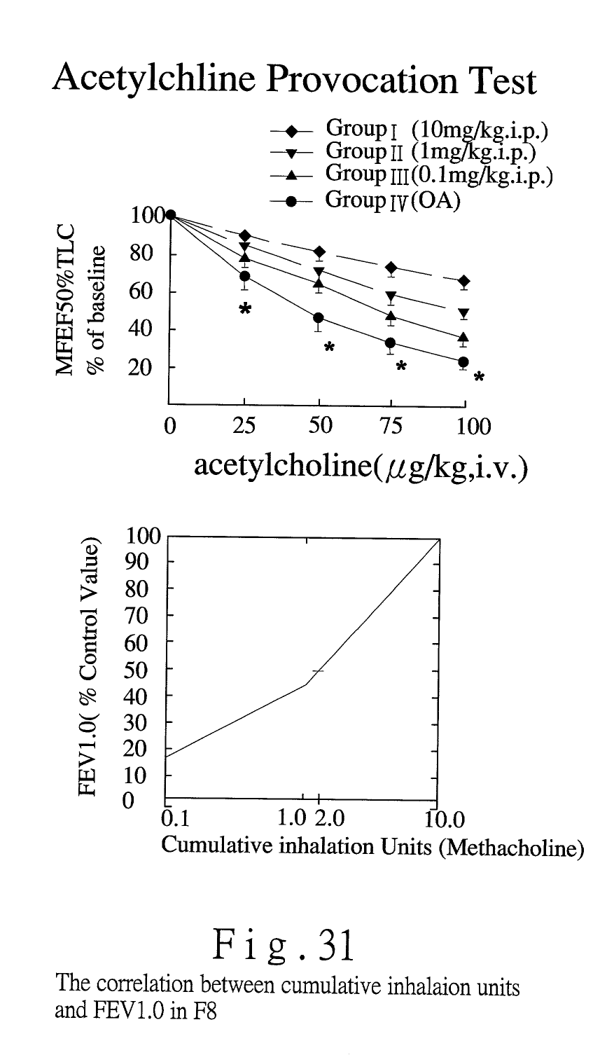

In summary, active fractions F4 and active compound F8 are isolated from Cordyceps sinensis in this invention, which can prophylactically and therapeutically improve the exacerbation of obstructive pulmonary function on late phase response of asthma in vivo, enhance Th1 cytokines expression, decrease Th2 cytokines and INOS gene mRNA expression, improve pulmonary histological change and block further progress of lung injury. There is also no acute toxicity of F-4. The activity of F4 and compound F8, which is purified from F-4, to inhibit platelets aggregation induced by PAF is shown in Table 12, the improvement of pulmonary function is shown in FIG. 31. It is suggested that the dosage of F8 about 3 mg/kg intramuscular injection will have therapeutic effect on prevention or treatment of human asthma by above data.

TABLE 1
Comparison at baseline of mean peak flow, MFEF 75%,
MFEF 50%, MFEF 25%, total dynamic lung compliance (Cdy),
airway opening pressure (PaO) at tidal breathing and total
lung capacity among group I, group II and group III

| | Group I | Group II | Group III |
|---|---|---|---|
| Peak flow, mL/sec | 104.8 ± 4.9 | 103.2 ± 5.6 | 105.6 ± 4.7 |
| MFEF 75%, mL/sec | 100.7 ± 4.6 | 100.3 ± 4.2 | 101.5 ± 4.9 |
| MFEF 50%, mL/sec | 80.8 ± 4.3 | 73.2 ± 5.1* | 82.7 ± 4.6 |
| MFEF 25%, mL/sec | 38.4 ± 4.3 | 32.3 ± 4.8* | 40.3 ± 4.5 |
| PaO, cmH₂O | 4.1 ± 0.6 | 4.1 ± 0.5 | 4.3 ± 0.6 |
| Cdyn, mL/cm H₂O | 0.51 ± 0.05 | 0.49 ± 0.06 | 0.53 ± 0.05 |
| Total lung capacity, | 15.3 ± 1.3 | 15.6 ± 1.6 | 15.4 ± 1.5 |
| mL | | | |

Anova test was used. *: p < 0.05 (comparison among group I, group II and group III).

TABLE 2
Comparison at baseline of mean peak flow, MFEF 75%,
MFEF 50%, MFEF 25%, total dynamic lung compliance (Cdy),
airway opening pressure (PaO) at tidal breathing and total
lung capacity between group I, group II and group III

| | Group I | Group II | Group III |
|---|---|---|---|
| Peak flow, mL/sec | 93.2 ± 5.6 | 95.6 ± 4.7 | 96.2 ± 4.7 |
| MFEF 75%, mL/sec | 80.3 ± 4.2 | 81.5 ± 4.9 | 81.7 ± 4.9 |
| MFEF 50%, mL/sec | 62.3 ± 5.1 | 61.1 ± 4.6 | 62.7 ± 4.6 |
| MFEF 25%, mL/sec | 33.9 ± 4.8* | 40.3 ± 4.5 | 41.9 ± 4.5 |
| PaO, cmH₂O | 3.2 ± 0.5 | 3.3 ± 0.6 | 3.5 ± 0.6 |
| Cdyn, mL/cm H₂O | 0.49 ± 0.06* | 0.53 ± 0.05 | 0.53 ± 0.05 |
| Total lung capacity, | 13.9 ± 1.6 | 13.4 ± 1.5 | 12.9 ± 1.5 |
| mL | | | |

Anova test was used. *: p < 0.05 (comparison among group I, group II and group III).

TABLE 3
Total differential cell counts in
bronchoalveolar lavage fluid in BNR.

| | Group I | Group II | Group III |
|---|---|---|---|
| Total cell count(×10⁴)/mL | 10.2 ± 2.5 | 15.4 ± 3.4* | 7.8 ± 2.3 |
| Macrophage | | | |
| Cell count (×10⁴)/mL | 7.77 ± 0.58 | 5.9 ± 0.71 | 6.86 ± 0.53 |
| Percentage of total cell | 76.2 ± 4.1 | 47.6 ± 5.7* | 88 ± 3.8 |
| count (%) | | | |
| Lymphocyte | | | |
| Cell count (×10⁴)/mL | 1.16 ± 0.18 | 2.77 ± 0.56* | 0.51 ± 0.12 |
| Percentage of total cell | 11.4 ± 2.6 | 22.3 ± 4.5* | 6.5 ± 1.6 |
| count (%) | | | |
| Neutrophil | | | |
| Cell count (×10⁴)/mL | 0.22 ± 0.3 | 0.22 ± 0.6 | 0.18 ± 0.3 |
| Percentage of total cell | 2.2 ± 0.4 | 1.8 ± 0.5 | 2.30 ± 0.4 |
| count (%) | | | |
| Eosinophil | | | |
| Cell count (×10⁴)/mL | 1.04 ± 0.3 | 3.51 ± 0.5* | 0.25 ± 0.2 |
| Percentage of total cell | 10.2 ± 2.8 | 28.3 ± 4.2* | 3.2 ± 2.2 |
| count (%) | | | |

Anova test was used. *: p < 0.05 (comparison among group I, group II and group III).

TABLE 4
Total differential cell counts in bronchoalveolar lavage fluid.

| | Group I | Group II |
|---|---|---|
| Total cell count (×10⁴)/mL | 93.7 ± 1.61* | 5.31 ± 0.58 |
| Macrophage | | |
| Cell count (×10⁴)/mL | 5.9 ± 0.71 | 6.22 ± 0.53 |
| Percentage of total cell count (%) | 47.6 ± 5.7* | 79.8 ± 3.8 |
| Lymphocyte | | |
| Cell count (×10⁴)/mL | 2.76 ± 0.56* | 0.66 ± 0.12 |
| Percentage of total cell count (%) | 22.3 ± 4.5* | 8.5 ± 1.6 |
| Neutrophil | | |
| Cell count (×10⁴)/mL | 0.22 ± 0.6 | 0.18 ± 0.3 |
| Percentage of total cell count (%) | 1.8 ± 0.5 | 2.30 ± 0.40 |
| Eosinophil | | |
| Cell count (×10⁴)/mL | 3.51 ± 0.5* | 0.73 ± 0.3 |
| Percentage of total cell count (%) | 28.3 ± 4.2* | 9.4 ± 2.2 |

Anova test was used. *: p < 0.05 (comparison among group I, group II and group III).

TABLE 5
Comparison of mean peak flow, MFEF 75%, MFEF 50%,
MFEF 25%, total dynamic lung compliance (Cdyn), airway
opening pressure (PaO) at tidal breathing and mean vital
capacity among group I, group II and group III

| | Group I | Group II | Group III |
|---|---|---|---|
| Peak flow, mL/sec | 23.59 ± 2.37 | 22.64 ± 2.07 | 23.64 ± 2.64 |
| MFEF 75%, mL/sec | 21.19 ± 2.28 | 19.94 ± 2.53 | 21.37 ± 2.20 |
| MFEF 50%, mL/sec | 16.78 ± 1.38 | 14.58 ± 1.31* | 17.55 ± 1.66 |
| MFEF 25%, mL/sec | 8.13 ± 1.23 | 6.06 ± 1.59* | 8.65 ± 1.17 |
| FEV 0.1, mL | 1.24 ± 0.16 | 1.26 ± 0.17 | 1.22 ± 0.13 |
| Cdyn, mL/cmH₂O | 0.42 ± 0.05 | 0.39 ± 0.04 | 0.44 ± 0.05 |
| PaO, cmH₂O | 3.5 ± 0.6 | 3.8 ± 0.5 | 3.4 ± 0.6 |
| TLC, mL | 1.52 ± 0.22 | 1.68 ± 0.25 | 1.49 ± 0.24 |
| Vital capacity, mL | 1.22 ± 0.15 | 1.29 ± 0.11 | 1.21 ± 0.16 |

Anova test was used. *: p < 0.05 (comparison among group I, group II and group III).

TABLE 6
Total differential cell counts in BAL fluid of BALBc
mice among group I, group II and group III.

| | Group I | Group II | Group III |
|---|---|---|---|
| Total cell count(×10⁴)/ | 2.12 ± 0.72* | 4.42 ± 1.13* | 1.27 ± 0.24 |
| mL | | | |
| Macrophage | | | |
| Cell count(×10⁴)/mL | 1.77 ± 0.1* | 2.86 ± 0.3* | 1.17 ± 0.04 |
| Percentage of total cell | 83.5 ± 3.9* | 64.7 ± 5.7* | 92.3 ± 2.8 |
| count (%) | | | |
| Lymphocyte | | | |
| Cell count(×10⁴)/mL | 0.11 ± 0.05* | 0.41 ± 0.1* | 0.04 ± 0.01 |
| Percentage of total cell | 5.1 ± 1.8* | 9.2 ± 2.4* | 3.1 ± 1.2 |
| count (%) | | | |
| Neutrophil | | | |
| Cell count(×10⁴)/mL | 0.11 ± 0.03* | 0.34 ± 0.06* | 0.05 ± 0.01 |
| Percentage of total cell | 5.2 ± 1.1* | 7.8 ± 1.3* | 3.9 ± 0.8 |
| count (%) | | | |
| Eosinophil | | | |
| Cell count(×10⁴)/mL | 0.13 ± 0.05* | 0.81 ± 0.14* | 0.01 ± 0.01 |
| Percentage of total cell | 6.2 ± 1.8* | 18.3 ± 3.7* | 0.7 ± 0.6 |
| count (%) | | | |

Anova test was used. *: p < 0.05 (comparison among group I, group II and group III).

TABLE 7
Comparison at baseline of mean peak flow,
MFEF 75% , MFEF 50%, MFEF 25%, total dynamic lung
compliance (Cdy), airway opening pressure (PaO) at
tidal breathing and total lung capacity among
group I, group II and group III

| | Group I | Group II | Group III |
|---|---|---|---|
| Peak flow, mL/sec | 105.6 ± 4.7 | 107.8 ± 5.2 | 96.2 ± 4.7 |
| MFEF 75%, mL/sec | 101.5 ± 4.9 | 106.4 ± 5.1 | 81.7 ± 4.9 |
| MFEF 50%, mL/sec | 75.7 ± 4.6* | 84.3 ± 4.9 | 62.7 ± 4.6 |
| MFEF 25%, mL/sec | 35.3 ± 4.5* | 46.3 ± 4.6 | 41.9 ± 4.5 |
| PaO, cmH₂O | 4.3 ± 0.6 | 4.1 ± 0.5 | 3.5 ± 0.6 |
| Cdyn, mL/cm H₂O | 0.53 ± 0.05 | 0.55 ± 0.06* | 0.53 ± 0.05 |
| Total lung capacity, mL | 15.4 ± 1.5 | 15.6 ± 1.6 | 12.9 ± 1.5 |

Anova test was used. *: p < 0.05 (comparison among group I, group II and group III).

TABLE 8
Total differential cell counts in
bronchoalveolar lavage fluid in BNR.

| | Group I | Group II | Group III |
|---|---|---|---|
| Total cell count(×10⁴)/mL | 14.8 ± 3.2* | 9.3 ± 2.4 | 6.7 ± 2.2 |
| Macrophage | | | |
| Cell count (×10⁴)/mL | 6.85 ± 0.64 | 6.32 ± 0.57 | 5.78 ± 0.55 |
| Percentage of total cell | 46.3 ± 5.5* | 68.0 ± 4.2 | 86.3 ± 3.9 |
| count (%) | | | |
| Lymphocyte | | | |
| Cell count (×10⁴)/mL | 3.37 ± 0.51* | 1.32 ± 0.17 | 0.46 ± 0.09 |
| Percentage of total cell | 22.8 ± 4.5* | 14.2 ± 2.1 | 6.9 ± 1.6 |
| count (%) | | | |
| Neutrophil | | | |
| Cell count (×10⁴)/mL | 0.31 ± 0.4 | 0.32 ± 0.2 | 0.15 ± 0.2 |
| Percentage of total cell | 2.1 ± 0.5 | 3.4 ± 0.5 | 2.2 ± 0.5 |
| count (%) | | | |
| Eosinophil | | | |
| Cell count (×10⁴)/mL | 4.26 ± 0.5* | 1.34 ± 0.4 | 0.31 ± 0.2 |
| Percentage of total cell | 28.8 ± 4.5* | 14.4 ± 2.5 | 4.6 ± 1.9 |
| count (%) | | | |

Anova test was used. *: p < 0.05 (comparison among group I, group II and group III).

TABLE 9
Comparison of mean peak flow, MFEF 75%, MFEF 50%,
MFEF 25%, total dynamic lung compliance (Cdyn), airway
opening pressure (PaO) at tidal breathing and mean vital
capacity among group I, group II and group III

| | Group I | Group II | Group III |
|---|---|---|---|
| Peak flow, mL/sec | 22.64 ± 2.07 | 23.24 ± 2.43 | 23.64 ± 2.64 |
| MFEF 75%, mL/sec | 19.94 ± 2.53 | 20.33 ± 2.31 | 21.37 ± 2.20 |
| MFEF 50%, mL/sec | 14.58 ± 1.31* | 16.48 ± 1.54 | 17.55 ± 1.66 |
| MFEF 25%, mL/sec | 6.06 ± 1.59* | 8.42 ± 1.48 | 8.65 ± 1.17 |
| FEV 0.1, mL | 1.26 ± 0.17 | 1.24 ± 0.15 | 1.22 ± 0.13 |
| Cdyn, mL/cmH₂O | 0.42 ± 0.04 | 0.44 ± 0.06 | 0.44 ± 0.05 |
| PaO, cmH₂O | 3.8 ± 0.5 | 3.6 ± 0.6 | 3.4 ± 0.6 |
| TLC, mL | 1.68 ± 0.25 | 1.46 ± 0.28 | 1.49 ± 0.24 |
| Vital capacity, mL | 1.29 ± 0.11 | 1.22 ± 0.18 | 1.21 ± 0.16 |

Anova test was used. *: p < 0.05 (comparison among group I, group II and group III).

TABLE 10
Total differential cell counts in BAL fluid of BALBc mice.

| | Group I | Group II | Group III |
|---|---|---|---|
| Total cell count(×10⁴)/ | 2.23 ± 0.64* | 4.42 ± 1.13* | 1.27 ± 0.24 |
| mL | | | |
| Macrophage | | | |
| Cell count (×10⁴)/ | 1.82 ± 0.21* | 2.86 ± 0.3* | 1.17 ± 0.04 |
| mL | | | |
| Percentage of total cell | 84.2 ± 3.4* | 64.7 ± 5.7* | 92.3 ± 2.8 |
| count (%) | | | |
| Lymphocyte | | | |
| Cell count (×10⁴)/ | 0.10 ± 0.04* | 0.41 ± 0.1* | 0.04 ± 0.01 |
| mL | | | |
| Percentage of total cell | 5.2 ± 1.6* | 9.2 ± 2.4* | 3.1 ± 1.2 |
| count (%) | | | |
| Neutrophil | | | |
| Cell count (×10⁴)/ | 0.13 ± 0.03* | 0.34 ± 0.06* | 0.05 ± 0.01 |
| mL | | | |
| Percentage of total cell | 5.1 ± 1.0* | 7.8 ± 1.3* | 3.9 ± 0.8 |
| count (%) | | | |
| Eosinophil | | | |
| Cell count (×10⁴)/ | 0.12 ± 0.04* | 0.81 ± 0.14* | 0.01 ± 0.01 |
| mL | | | |
| Percentage of total cell | 6.3 ± 1.6* | 18.3 ± 3.7* | 0.7 ± 0.6 |
| count (%) | | | |

Anova test was used. *: p < 0.05 (comparison among group I, group II and group III).

TABLE 12
The inhibitory effects of Cordyceps sinensis active
fraction F-4 and active component F-8 on platelet
aggregation induced by PAF.

| | Dosage | | Inhibitory Activity (%) |
|---|---|---|---|
| F-4 | 500 | [symbol] g/ml | 83.8 ± 9.1 |
| F-8 | 0.625 | mM | 97 ± 2.6 |

1. A method for isolating a pulmonary function-improving fraction from Cordyceps sinensis, comprises oven drying and grinding fruiting body of Cordyceps sinensis, extracting with organic solvents, concentrating extracts, and isolating said active fractions by silica gel column chromatography in conjunction with in vitro assay of inhibition of PAF induced rabbit platelet aggregation, histopathological change and Th1 cytokines gene expression.

2. A method as in claim 1, wherein said organic solvent for extraction is selected from the group consisting of methanol, acetone, ethyl acetate, chloroform and methylene chloride.

3. A method as in claim 2, wherein said organic solvent is methanol and ethyl acetate.

4. A method as in claim 1, wherein said silica gel chromatography is carried out by eluting with mixture of n-hexane, ethyl acetate and methanol in different volume ratio.

5. A pulmonary function-improving fraction from Cordyceps sinensis, characterized in that said active fraction is obtainable by the method according to any one of claim 1-4 and in that it can suppress platelet activating factor (PAF) inducing rabbit platelet aggregation in vitro, improving pulmonary function of animals with ovalbumin (OA) induced bronchial hyperresponsiveness in vivo, enhancing Th1 cytokines that inhibit Th2 cytokines and induce nitric oxide synthase (iNOS) genes expression in vivo in brown Norway rats (BNR) with OA induced bronchial hyperresponsiveness, alleviating the bronchial hyperresponsiveness of bronchial asthma and histophathologically preventing chronic inflammatory injury.

6. A method for isolating a pulmonary function-improving compound from Cordyceps sinensis, comprises subjecting an active fraction according to claim 5 to silica gel column chromatography by eluting with methylene chloride-methanol (100:1 to 1:1 (v/v)) in conjunction with identifying said active compound by in vitro assay of inhibition of PAF induced rabbit platelet aggregation, histophathological change and Th1 cytokines gene expression.

7. A method as in claim 6, wherein said active compound is isolated by said silica gel column chromatography using methylene chloride-methanol 10:1 (v/v) as the eluting solvent.

8. A pulmonary function-improving compound from Cordyceps sinensis, characterized in that it is [(24R)-ergosta-7,22-diene-3β,5α,6 β-triol] of a structural formula.

9. Use of the active fraction from Cordyceps sinensis according to claim 5 for suppressing platelet activating factor (PAF) inducing rabbit platelet aggregation, improving pulmonary function of animals with ovalbumin (OA) induced bronchial hyperresponsiveness, enhancing Th1 cytokines that inhibit Th2 cytokines and induce nitric oxide synthase (iNOS) genes expression in vivo in brown Norway rats (BNR) with OA induced bronchial hyperresonsive, allevating the bronchial hyperresponsiveness of bronchial asthma and histophathologically preventing chronic inflammatory injury.

10. Use of the active compound from Cordyceps sinensis according to claim 8 for suppressing platelet activating factor (PAF) inducing rabbit platelet aggregation, improving pulmonary function of animals with ovalbumin (OA) induced bronchial hyperresponsiveness, enhancing Th1 cytokines that inhibit Th2 cytokines and induce nitric oxide synthase (iNOS) genes expression in vivo in brown Norway rats (BNR) with OA induced bronchial hyperresponsiveness, alleviating the bronchial hyperresponsiveness of bronchial asthma and histopathologically preventing chronic inflammatory injury.
